(19) United States
(12) Patent Application Publication
(10) Pub. No.: US 2025/0215407 A1
(43) Pub. Date: Jul. 3, 2025

(54) IMPROVED MACROMOLECULES AND METHODS FOR DESIGNING SAME

(71) Applicants: RAMOT AT TEL-AVIV UNIVERSITY LTD., Tel Aviv (IL); MOR RESEARCH APPLICATIONS LTD, Ramat Gan (IL)

(72) Inventors: Daniel OFFEN, Tel Aviv (IL); Roy RABINOWITZ, Tel Aviv (IL); Felix BENNINGER, Tel Aviv (IL); Oded SHOR, Tel Aviv (IL)

(21) Appl. No.: 18/853,261

(22) PCT Filed: Apr. 4, 2023
(86) PCT No.: PCT/IL2023/050363

§ 371 Date: Oct. 1, 2024

Related U.S. Application Data
(60) Provisional application No. 63/327,918, filed on Apr. 6, 2022.
(60) Provisional application No. 63/327,928, filed on Apr. 6, 2022.

(51) Int. Cl. C12N 9/22; C12N 15/52; G16B 15/30; G16B 20/00

(52) U.S. Cl. CPC C12N 9/22; C12N 15/52; G16B 15/30; G16B 20/00

(57) ABSTRACT

The present invention is directed to, inter alia, a method for identifying a reference macromolecule for which a variant having improved function can be identified and/or engineered. Further provided is a macromolecule variant, such as, but not limited to Cas protein variant(s), being characterized by having improved function compared to a reference, and a method for designing same.

### CROSS-REFERENCE TO RELATED APPLICATIONS

This application claims the benefit of priority of U.S. Provisional Patent Application No. 63/327,918, titled “ENZYMES AND METHODS FOR DESIGNING SAME”, filed Apr. 6, 2022, and U.S. Provisional Patent Application No. 63/327,928, titled “MACROMOLECULES AND METHODS FOR DESIGNING SAME”, filed Apr. 6, 2022. The contents of which are all incorporated herein by reference in their entirety.

### REFERENCE TO AN ELECTRONIC SEQUENCE LISTING

The contents of the electronic sequence listing (RMT-MOR-P-017-PCT.xml; size: 65,619 bytes; and date of creation: Apr. 4, 2023) is herein incorporated by reference in its entirety.

### FIELD OF INVENTION

The present invention is in the field of engineering of biological molecules, and specifically relates to methods enabling performance of in silico macromolecules e.g., protein, DNA, and RNA, engineering.

### BACKGROUND

The clustered regularly interspaced short palindromic repeats (CRISPR)-CRISPR-associated protein (Cas) system is a prokaryotic adaptive immune system, conferring immunity against bacteriophages and plasmids based on nucleic acids recognition. It has been employed as a gene-editing tool in eukaryotic cells owing to its unique RNA-guided targeting attributes. Class 2 CRISPR systems consist of a single Cas effector protein. Upon binding to a guide-RNA (gRNA) molecule, it directs the Cas protein toward its target sequence DNA or RNA, depending on the type and subtype of the Cas protein. Target recognition is mediated by base pairing between the gRNA and the target sequence. For the commonly studied Streptococcus pyogenes (Spy)Cas9, the gRNA base-pairs with the target strand DNA (TS-DNA), a stage that drives a conformational transformation of Cas9, leading it to cleave the target DNA. Recently, the field of gene-editing has entered a new era, as the CRISPR-Cas system was introduced into patients cells, ex vivo and in vivo, and reportedly contributing positive results in clinical outcomes. Understanding the accuracy and specificity of CRISPR-Cas9 is essential to better design and develop improved future gene-editing therapeutics. Previous studies have revealed the protein structure of SpyCas9, paving the way to structural investigation of the protein and its functions.

Normal mode analysis (NMA) is a computational method that can assess which conformational variations are accessible for a given protein. It relies on the premise that a protein is an oscillating system. Coarse-grained NMA overcomes the computationally limiting factor of analyzing large numbers of atoms in a protein by representing each residue using a single atom—the Cα.

NMA was shown to provide structural and dynamic details on the mechanism of action of Streptococcus pyogenes (Spy)Cas9. Nevertheless, it was not utilized to study the sequence-dependent activity of enzymatic systems, e.g., the CRISPR-Cas system.

There is a need for methods for engineering proteins and other macromolecules with improved functionally, specifically proteins interacting with nucleic acids.

### SUMMARY

According to some embodiments, there is provided a method for identifying a reference macromolecule for which a macromolecule variant having improved function can be engineered.

According to some embodiments, there is provided a method for engineering a macromolecule variant having improved function as compared to a reference macromolecule.

The present invention, in some embodiments, is based, in part, on the findings that support the relevance of NMA to study the function of proteins, e.g., SpyCas9, and suggest a new approach to predict on-target and off-target activity and specificity of enzymes, including but not limited to, CRISPR-Cas systems.

According to a first aspect, there is provided a method for identifying a reference macromolecule for which a variant having improved function can be engineered, the method comprising: (a) receiving a dataset comprising data on relative functionality of: (i) variants of the reference macromolecule comprising an altered residue or moiety; (ii) the reference macromolecule in complex with different binding counterparts; or (iii) both; (b) in silico calculating a value of entropy for the variants of the reference macromolecule and/or the reference macromolecule in complex with different binding counterpart of the received dataset; (c) determining a correlation value between the calculated values of entropy and the received relative functionality data; wherein a correlation value above a predetermined threshold indicates a true correlation between entropy and function; and (d) identifying a reference macromolecule with a true correlation between entropy and function as a reference macromolecule for which a variant having improved function can be engineered.

According to another aspect, there is provided a method for engineering a variant of a macromolecule having improved function as compared to a reference macromolecule, the method comprising: (a) identifying a reference macromolecule suitable for engineering by the method disclosed herein; (b) generating a standard curve of entropy value to function based on the calculated entropy values and the received relative functionality data; (c) in silico calculating a value of entropy for at least one new variant of the reference macromolecule; (d) based on the generated standard curve and the calculated value of entropy predicting relative function of the at least one new variant; and (e) selecting a new variant with predicted improved relative function as compared to the reference macromolecule; thereby engineering a variant of a macromolecule having improved function as compared to a reference macromolecule.

According to another aspect, there is provided a macromolecule variant engineered or synthesized according to the method disclosed herein.

According to another aspect, there is provided Cas variant protein of a reference Cas protein wherein the reference Cas protein comprises an amino acid sequence as set forth in (SEQ ID NO: 1), wherein the variant protein comprises at least one amino acid substitution in a position selected from the group consisting of: 692, 1129, 1231, 9, 519, 177, 381, 395, 414, 512, 538, 539, 735, 739, 743, 758, 871, 1256, 1283 1359, and any combination thereof, in the reference Cas protein.

According to another aspect, there is provided a nucleic acid molecule comprising a nucleic acid sequence encoding the Cas variant protein disclosed herein.

According to another aspect, there is provided an expression vector comprising the nucleic acid sequence disclosed herein.

According to another aspect, there is provided a cell comprising any one of: (a) the Cas variant protein disclosed herein; (b) the nucleic acid sequence disclosed herein; (c) the expression vector disclosed herein; or (d) any combination of (a) to (c).

According to another aspect, there is provided a composition comprising any one of: (a) the Cas variant protein disclosed herein; (b) the nucleic acid sequence disclosed herein; (c) the expression vector disclosed herein; (d) the cell disclosed herein; or (e) any combination of (a) to (d), and an acceptable carrier.

According to another aspect, there is provided a method for modifying at least one target nucleic acid sequence of interest, the method comprising contacting the at least one target nucleic acid sequence of interest with an effective amount of: (a) any one of: (i) the Cas variant protein disclosed herein; (ii) the nucleic acid sequence disclosed herein; (iii) the expression vector disclosed herein; and (iv) any combination of (i) to (iii); and (b) at least one target recognition element or a nucleic acid sequence encoding thereof, thereby modifying the at least one target nucleic acid sequence of interest.

In some embodiments, the dataset of step (a) comprises data on relative functionality of the reference macromolecule in complex with different binding counterparts and the method further comprises as part of step (b) in silico calculating a value of entropy for the different binding counterparts in complex with the reference macromolecule and as part of step (c) determining a correlation value between the calculated value of entropy of the different binding counterparts and the received functionality data, wherein a correlation value of the macromolecule entropy above a predetermined threshold and a correlation value of the binding counterpart entropy above a predetermined thresholds indicates a true correlation.

In some embodiments, the dataset of step (a) comprises data on relative functionality of the reference macromolecule in complex with different binding counterparts and the method further comprises: receiving a second dataset comprising data on relative functionality of variants of the reference macromolecule comprising an altered residue or moiety, in silico calculating a value of entropy for the variants of the reference macromolecule, determining a correlation value between the calculated values of entropy of the altered macromolecules and the relative functionality data of the second dataset; and wherein step (d) comprises selecting a reference macromolecule with true correlation based on the dataset of step (a) and the second dataset.

In some embodiments, the correlation is a Pearson correlation coefficient (R).

In some embodiments, the above a predetermined threshold is above an R of 0.55.

In some embodiments, the calculating a value of entropy comprises normal mode analysis (NMA).

In some embodiments, the correlation is a positive correlation, or a negative correlation and the correlation value is an absolute value of the correlation.

In some embodiments, the value of entropy is an absolute value of entropy.

In some embodiments, the macromolecule is a protein, a polynucleotide, or a complex comprising both.

In some embodiments, the macromolecule is a protein, and wherein the functionality is selected from the group consisting of: thermostability, conformational transition, binding to a substrate, enzymatic activity, and signaling.

In some embodiments, the protein is an enzyme, the functionality in enzymatic activity or binding and the substrate is a target of the enzymatic activity.

In some embodiments, the binding counterpart is a protein or nucleic acid molecule that forms a complex with the reference macromolecule by direct binding or binding via an intermediate molecule.

In some embodiments, the intermediate molecule is a nucleic acid molecule that binds a binding counterpart that is a nucleic acid molecule.

In some embodiments, the binding counterpart, the intermediate molecule, or both is a nucleic acid molecule selected from DNA and RNA.

In some embodiments, the protein is a genome-editing protein, optionally wherein the genome-editing protein is a CRISPR associated (Cas) protein.

In some embodiments, the binding counterpart is a target genomic locus, the intermediate molecule is a guide RNA (gRNA) and the value of entropy is calculate for any one of: the Cas protein alone, the Cas protein complexed with a gRNA and the Cas protein complexed with a gRNA and the target genomic locus.

In some embodiments, the method further comprises synthesizing the selected macromolecule variant engineered to have improved function as compared to the reference macromolecule.

In some embodiments, the method further comprises determining the synthesized macromolecule variant has improved function as compared to the reference macromolecule, wherein the determining is performed in vitro, in vivo, ex vivo, or any combination thereof.

In some embodiments, the macromolecule is a protein, a polynucleotide, or a complex comprising at least one protein and at least one polynucleotide.

In some embodiments, the polynucleotide comprises DNA, RNA, or a hybrid thereof.

In some embodiments, the reference Cas protein is a Streptococcus pyogenes Cas wildtype (SpyCas) protein.

In some embodiments, the SpyCas is SpyCas9.

In some embodiments, the Cas variant protein is characterized by having improved function compared to the Cas reference protein.

In some embodiments, the improved function is selected from improved substrate specificity and improved nuclease activity.

In some embodiments, the at least one amino acid substitution is selected from the group consisting of: K1129T, K1231I, L9R, N692V, T519V, D177A, E381N, R395A, I414A, S512R, A538I, F539V, K735W, Q739R, V743N, N758V, P871I, Q1256W, A1283E, R1359Q, and any combination thereof.

In some embodiments, the at least one amino acid substitution is selected from the group consisting of: K1129T, K1231I, L9R, N692V, T519V, and any combination thereof.

In some embodiments, the composition is a pharmaceutical composition.

In some embodiments, the target nucleic acid sequence of interest is in cell of a subject, and the contacting is administering a therapeutically effective to the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a non-limiting general scheme of normal mode analysis (NMA) that predicts the activity and specificity in a sequence-dependent manner. NMA yields entropy scores that correlate with empiric SpyCas9 activity data. Modifications were made to all parts of the structure: protein (high-fidelity variants mutations), DNA (four different EMX1 sites) and sgRNA (mismatches assay) while retaining high correlations. PDB: 5F9R.

FIGS. 2A-2D include heatmaps and graphs showing SpyCas empiric activity and structure-based entropy. (2A) Heatmap representations of previously reported empiric SpyCas9 activity (specificity measured as the ratio of mismatch/perfect match), the entropy of the DNA and the SpyCas9 protein (log (|ΔG|)) in the presence of single-base mismatches in four loci within the EMX1 gene. The color scale bar orientation is determined by the direction of the correlation (positive/negative). (2B) Correlations between the empiric activity (x) and the |ΔG| of the DNA (y). (2C) Correlations between the empiric activity (x) and the |ΔG| of the protein (y). (2D) Correlations between the |ΔG| of the DNA (x) and the |ΔG| of the protein (y). All correlation plots are shown with a 95% confidence interval and P-value<0.00005 (N=57). The correlation values represent the Pearson correlation coefficient.

FIGS. 3A-3B include graphs and 3-dimensional structures showing the correlation between the empiric activity in the presence of mismatches and the entropy of each amino acid in the structure of SpyCas9 for each mismatch. (3A) Absolute values of the Pearson correlation coefficient R, measured in all amino acids along with the structure of SpyCas9 in the presence of mismatches in four genomic loci. The measured entropy relates to the α-carbon of each amino acid. The dashed line represents a threshold of R=0.55. Regions containing residues with R greater than the threshold in more than one site are marked in light blue. The 2D representation of the protein domains shows the regions in which the entropy of the amino acids best correlate with the empiric activity data. Scale range 0<R<0.8. (3B) The structure of SpyCas9 highlighting the residues with R>0.55 (mesh). Colors indicate the number of sites (1-3) in which the R value for this residue crossed the threshold (left). The right panel is a 3D representation of the protein domains. The target strand DNA (TS-DNA), non-target strand (NTS-DNA) and the sgRNA are represented as simplified lines, while the protein is visualized as a cartoon.

FIGS. 4A-4D include heatmaps and a graph showing that NMA predicts and replicates specificity and activity of eight SpyCas9 variants with improved. (4A) Entropy profile heatmaps of SpyCas9 variants in the presence of gRNA mismatches at the EMX1—site3 locus (log(|ΔG|) measured at the DNA molecule (chain C—TS-DNA). (4B) Average activity and specificity scores as previously reported and determined by the TTISS method. (4C) Correlation between the activity score of each variant and its corresponding average entropy score (log(|ΔG|). The correlation plot is shown with a 95% confidence interval and P-value=0.024123 (N=9). (4D) The Pearson correlation coefficient (R) of each position within the gRNA, representing the feasibility of each position to predict the activity outcome (average per variant) using the entropy score (average per position per variant). #=0.06<P-value<0.05, *=P-value<0.05, **=P-value<0.005.

FIG. 5 includes a flowchart demonstrating, as a non-limiting example, the steps for identifying or designing a macromolecule variant having improved function compared to a reference, according to some embodiments of the invention.

FIG. 6 includes a flowchart demonstrating, as a non-limiting example, the steps for engineering a macromolecule variant having improved function as compared to a reference, according to some embodiments of the invention.

FIG. 7 includes a flowchart demonstrating, as a non-limiting example, the steps for synthesizing a macromolecule variant having improved function as compared to a reference, according to some embodiments of the invention.

FIGS. 8A-8B include vertical bar graphs and heatmaps showing on-target activity and off-target analysis of the HF Cas9 variants. (8A) The on-target activity of WT SpyCas9 and the tested HF variants with three sgRNAs targeting EMX1, VEGFA and AAVS1. The activity is measured as the percentage of modified reads. The line shows the average activity of WT SpyCas9. Error bars describe the standard error of the mean. (8B) Off-targeting by WY SpyCas9 and the tested HF variants with the three sgRNAs. Each off-target (OT) site is represented in a row. The white-red scale represents the percentage of modified reads for each locus. Only OT sites with >1% modified reads are shown.

FIG. 9. Includes a general non-limiting scheme of the ComPE pipeline disclosed herein.

FIGS. 10A-10D include chromatograms and graphs showing that entropy values correlate with empirical activity and specificity. (10A) Activity-NMA correlation coefficient |r| was calculated for each of the residues of all seven proteins (WT+6 HF variants) to assess which residue provides the best correlation for all proteins. (10B) The Activity-NMA correlation plot describes the experimental activity values for each variant and the calculated entropy absolute values as measured at residue H641. The Pearson correlation coefficient is r=−0.9885. P value=3×10⁻⁵. (10C) Specificity-NMA correlation coefficient |r| was calculated for each of the residues of all seven proteins to assess which residue provides the best correlation for all proteins. (10D) The specificity-NMA correlation plot describes the experimental activity values for each variant and the calculated entropy absolute values as measured at residue K1107. The Pearson correlation coefficient is r=0.7402. P value=0.0571. All correlation plots are shown with a 95% confidence interval.

FIGS. 11A-11B include graphs showing in-silico deep mutational scanning of SpyCas9 variants and analyses of their activity and specificity. (11A) Empirical cumulative distribution function (ECDF) plot representing the proportion of variants and their log (|entropy|) values, based on the entropy score of residue H641 to assess the activity of all possible single-amino acid substitutions. The blue line represents the deep mutational scanning derivative variants (25,878 variants), while the dots represent WT (yellow) or the known HF Cas9 enzymes (light red) and the chosen candidates (green). The magnification box displays the sigmoid area of the curve. (11B) ECDF plot representing the proportion of variants and their log (|entropy|) values, based on the entropy score of residue K1107 to assess the specificity of all possible single-amino acid substitutions. The red line represents the deep mutational scanning derivative variants.

FIGS. 12A-12D include scheme of a non-limiting study design, sequences, and heat maps showing that GFP-disruption assay reveals the specificity profile of HF SpyCas9 variants. (12A) Description of the experimental workflow. (12B) gRNA sequences used in this assay. The perfect match gRNA has no mismatches, while all 19 other gRNAs have mismatches in each position of the gRNA (except the 20^{th} position). Positions are annotated as distance from protospacer-adjacent motif (PAM). The mismatch is marked in red lower-case letters. (12C) Heatmap representation of the GFP disruption by different Cas9 variants combined with mismatched gRNAs analyzed using flow cytometry. All values were calculated as the ratio of GFP disruption in the presence of a mismatch relative to the disruption with a perfect match gRNA. Thus, 100% indicates equal activity of the Cas9 variant combined with both the perfect match gRNA and in the presence of a mismatch. Values below 100% (towards the blue shades) indicate improved sensitivity to a mismatch in each position. Each data point represents the average of two biological replicates. (12D) Normalization of FIG. 12C to demonstrate the sensitivity of Cas9 variants compared to WT SpyCas9. Values around 1 indicate no change in mismatch sensitivity compared to WT SpyCas9. Above 1 (dark red shades), sensitivity is impaired, while below 1 (blue shades), mismatch sensitivity is improved compared to WT SpyCas9.

FIGS. 13A-13D include structural description of the mutated residues of the four HF SpyCas9 variants disclosed herein. (13A) S512; (13B) N692V; (13C) K735W; and (13D) V743N.

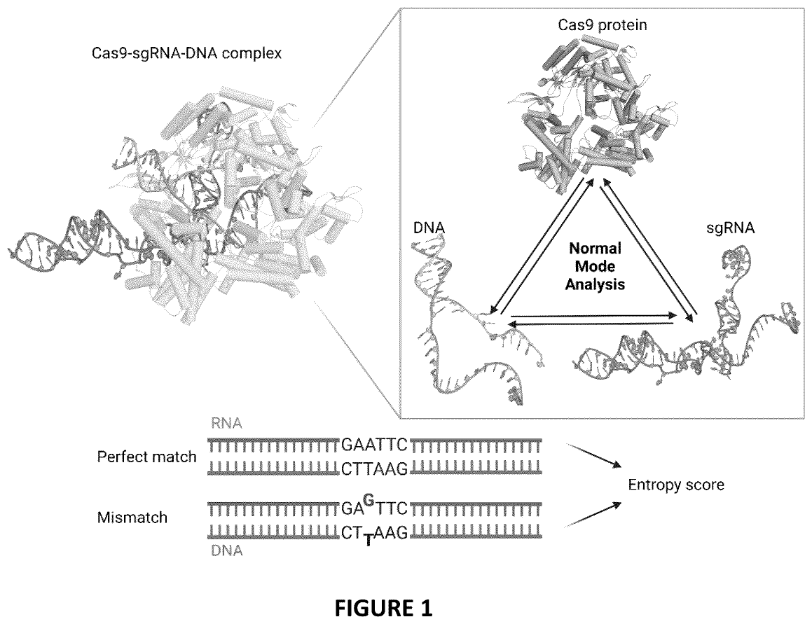

### DETAILED DESCRIPTION

### Methods

According to some embodiments, there method for identifying a reference macromolecule for which a variant having improved function can be engineered.

In some embodiments, the method comprises: (a) receiving a dataset comprising data on relative functionality of: (i) variants of the reference macromolecule comprising an altered residue or moiety; (ii) the reference macromolecule in complex with different substrates; or (iii) both; (b) in silico calculate a value of entropy for the variants of the reference macromolecule and/or the reference macromolecule in complex with different substrates of the received dataset; (c) determine a correlation value between the calculated values of entropy and the received relative functionality data; and (d) select a reference macromolecule with a true correlation between entropy and function as a reference macromolecule for which a variant having improved function can be engineered.

In some embodiments, a correlation value equal to or above a predetermined threshold indicates a true correlation between entropy and function.

In some embodiments, a correlation value below a predetermined threshold indicates no correlation between entropy and function.

In some embodiments, the dataset of step (a) comprises data on relative functionality of the reference macromolecule in complex with different substrates.

In some embodiments, the method further comprises as part of step (b) in silico calculating a value of entropy for the different substrates in complex with the reference macromolecule.

In some embodiments, the method further comprises as part of step (c) determining a correlation value between the calculated value of entropy of the different substrates and the received functionality data.

In some embodiments, any one of: a correlation value of the macromolecule entropy being equal to or above a predetermined threshold, and a correlation value of the substrate entropy being equal to or above a predetermined threshold indicates a true correlation.

In some embodiments, any one of: a correlation value of the macromolecule entropy being below a predetermined threshold, and a correlation value of the substrate entropy below a predetermined threshold indicates no correlation.

In some embodiments, the dataset of step (a) comprises data on relative functionality of the reference macromolecule in complex with different substrates.

In some embodiments, the method further comprises: receiving a second dataset comprising data on relative functionality of variants of the reference macromolecule comprising an altered residue or moiety, in silico calculating a value of entropy for the variants of the reference macromolecule and determining a correlation value between the calculated values of entropy of the altered macromolecules and the relative functionality data of the second dataset.

In some embodiments, step (d) comprises selecting a reference macromolecule with true correlation based on the dataset of step (a) and the second dataset.

In some embodiments, the functionality is selected from: thermostability, conformational transition, binding to a substrate, enzymatic activity, or signaling.

According to some embodiments, there is provided a method for engineering a macromolecule variant having improved function as compared to a reference macromolecule.

In some embodiments, the method comprises: (a) selecting a reference macromolecule suitable for engineering by the method disclosed herein; (b) generating a standard curve of entropy value to function based on the calculated entropy values and the received relative functionality data; (c) in silico calculate a value of entropy for at least one new variant of the reference macromolecule; (d) based on the generated standard curve and the calculated value of entropy predict relative function of the at least one new variant; and (e) select/identify a new variant with predicted improved relative function as compared to the reference macromolecule.

In some embodiments, the method, further comprises synthesizing the selected macromolecule variant engineered to have improved function as compared to the reference macromolecule.

In some embodiments, the method further comprises determining the synthesized macromolecule variant has improved function as compared to the reference macromolecule.

In some embodiments, the determining is performed in vitro, in vivo, ex vivo, or any combination thereof.

In some embodiments, the macromolecule comprises a protein. In some embodiments, the macromolecule comprises a polynucleotide.

In some embodiments, the macromolecule comprises a complex comprising a plurality of macromolecules. In some embodiments, the macromolecule comprises a complex comprising a plurality of types of macromolecules.

In some embodiments, a complex of macromolecules comprises at least one protein and at least one polynucleotide. In some embodiments, the at least one protein comprises a plurality of types of proteins, a plurality of molecules of the same protein, or both. In some embodiments, the at least one polynucleotide comprises a plurality of types of polynucleotides, a plurality of molecules of the same polynucleotide, or both. In some embodiments, a complex of macromolecules comprises at least two proteins. In some embodiments, a complex of macromolecules comprises at least two polynucleotides.

In some embodiments, a polynucleotide comprises DNA, RNA, or a hybrid thereof.

In some embodiments, DNA comprises genomic DNA, cDNA, or both.

In some embodiments, RNA comprises mRNA, signal guide RNA (sgRNA), double stranded RNA, short inhibiting RNA (siRNA), short hairpin RNA (shRNA), long non-coding RNA (lncRNA) or any combination thereof. In some embodiments, the RNA is a ribozyme. In some embodiments, the RNA is an rRNA. In some embodiments, the RNA is a tRNA.

According to some embodiments, there is provided a method for identifying a reference protein for which a protein variant having improved function can be engineered.

In some embodiments, the method comprises: (a) receiving a dataset comprising data on relative functionality of: (i) variants of the reference protein comprising an altered residue; (ii) the reference protein in complex with different substrates; or (iii) both; (b) in silico calculate a value of entropy for the variants of the reference protein and/or the reference protein in complex with different substrates of the received dataset; (c) determine a correlation value between the calculated values of entropy and the received relative functionality data; and (d) select/identify a reference protein with a true correlation between entropy and function as a reference protein for which a protein variant having improved function can be engineered.

In some embodiments, the dataset of step (a) comprises data on relative functionality of the reference protein in complex with different substrates and the method further comprises as part of step (b) in silico calculating a value of entropy for the different substrates in complex with the reference protein and as part of step (c) determining a correlation value between the calculated value of entropy of the different substrates and the received functionality data.

In some embodiments, the dataset of step (a) comprises data on relative functionality of the reference protein in complex with different substrates and the method further comprises: receiving a second dataset comprising data on relative functionality of variants of the reference protein comprising an altered residue, in silico calculating a value of entropy for the variants of the reference protein, and determining a correlation value between the calculated values of entropy of the altered proteins and the relative functionality data of the second dataset.

In some embodiments, step (d) comprises selecting a reference protein with true correlation based on the dataset of step (a) and the second dataset.

In some embodiments, correlation is a Pearson correlation coefficient (R).

In some embodiments, equal to or above a predetermined threshold is equal to or above an R of 0.55.

In some embodiments, calculating a value of entropy comprises normal mode analysis (NMA).

In some embodiments, calculating a value of entropy is determined using, or is based on, a quantitative kinetic model such as, but not limited to, the method(s) being disclosed in Eslami-Mossallam et al., 2022, Nature communication.

In some embodiments, correlation is a positive correlation or a negative correlation.

In some embodiments, correlation value is an absolute value of a correlation.

In some embodiments, a value of entropy is an absolute value of entropy.

In some embodiments, functionality is selected from: thermostability, conformational transition, binding to a substrate, enzymatic activity and signaling.

In some embodiments, the reference protein is selected from: enzyme, receptor, transporter, cell signaling protein, ligand binding protein, antibody, structural protein, peptide, aptamer, RNA-binding protein, DNA-binding protein, immunomodulator, hormone, or any combination thereof.

In some embodiments, the reference protein is an enzyme, the functionality is enzymatic activity or binding, and the substrate is a target of the enzymatic activity.

In some embodiments, the substrate is a protein or nucleic acid molecule that forms a complex with the reference protein by direct binding or binding via an intermediate molecule.

In some embodiments, an intermediate molecule comprises a nucleic acid molecule that binds a substrate that is a nucleic acid molecule.

In some embodiments, a substrate, an intermediate molecule, or both, comprises a nucleic acid molecule being a DNA, RNA, or a hybrid thereof.

In some embodiments, a reference protein is a genome-editing protein.

In some embodiments, a genome-editing protein comprises a CRISPR associated (Cas) protein.

In some embodiments, a substrate comprises a target genomic locus.

In some embodiments, an intermediate molecule comprises a guide RNA (gRNA).

In some embodiments, a value of entropy is calculated for a Cas protein alone, a Cas protein complexed with a gRNA, or a Cas protein complexed with a gRNA and a target genomic locus.

According to some embodiments, there is provided a method for engineering a protein variant having improved function as compared to a reference protein.

In some embodiments, the method comprises: (a) selecting a reference protein suitable for engineering by the method disclosed herein; (b) generating a standard curve of entropy value to protein function based on the calculated entropy values and the received relative functionality data; (c) in silico calculate a value of entropy for at least one new variant of the reference protein; (d) based on the generated standard curve and the calculated value of entropy predict relative protein function of the at least one new variant; and (e) select a new variant with predicted improved relative protein function as compared to the reference protein.

In some embodiments, the method further comprises synthesizing the selected protein variant engineered to have improved function as compared to the reference protein.

In some embodiments, the method further comprises determining the synthesized protein variant has improved function as compared to the reference protein.

According to one embodiment, the synthesized protein as disclosed herein may be synthesized or prepared by any method and/or technique known in the art for peptide synthesis.

According to another embodiment, the protein may be synthesized by a solid phase peptide synthesis method of Merrifield (see J. Am. Chem. Soc, 85:2149, 1964). According to another embodiment, the peptide of the invention can be synthesized using standard solution methods, which are well known in the art (see, for example, Bodanszky, M., Principles of Peptide Synthesis, Springer-Verlag, 1984).

In general, the synthesis methods comprise sequential addition of one or more amino acids or suitably protected amino acids to a growing peptide chain bound to a suitable resin. Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha,alpha)-dimethyl-3,5 dimethoxybenzyloxycarbonyl, o-nitrophenylsulfenyl, 2-cyano-t-butyloxycarbonyl, 9-fluorenylmethyloxycarbonyl (Fmoc) and the like. In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic peptide synthesizer as is well known in the art.

In another embodiment, a protein as disclosed herein may be synthesized such that one or more of the bonds, which link the amino acid residues of the peptide are non-peptide bonds. In another embodiment, the non-peptide bonds include, but are not limited to, imino, ester, hydrazide, semicarbazide, and azo bonds, which can be formed by reactions well known to one skilled in the art.

In some embodiments, a protein as disclosed herein may be synthesized as a recombinant protein in a compatible recombinant cell system or a cell free system.

Methods for synthesizing, purifying, isolating, retrieving, or any combination thereof, of a recombinant protein are common and would be apparent to one of ordinary skill in the art.

According to some embodiments, there is provided a protein variant having improved function as compared to the reference protein, identified and/or engineered according to the method disclosed herein.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

The terms “polynucleotide,” “polynucleotide sequence,” “nucleic acid sequence,” and “nucleic acid molecule” are used interchangeably herein. These terms encompass nucleotide sequences and the like. A polynucleotide may be a polymer of RNA or DNA that is single- or double-stranded, that optionally contains synthetic, non-natural, or altered nucleotide bases.

### Protein Variants, Cells, and Compositions

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 1), wherein the variant protein comprises at least one amino acid substitution in a position selected from: 1129, 1231, 9, 692, 519, 177, 381, 395, 414, 512, 538, 539, 735, 739, 743, 758, 871, 1256, 1283 1359, or any combination thereof, of the reference Cas protein.

According to some embodiments, there is provided a Cas variant protein of a reference Cas protein comprising the amino acid sequence: MDKKYSIGX₁DIGTNSVGWAVITDEYKVPSKKFKVLGNTDRHSIKKNLIGALLFDS GETAEATRLKRTARRRYTRRKNRICYLQEIFSNEMAKVDDSFFHRLEESFLVEEDK KHERHPIFGNIVDEVAYHEKYPTIYHLRKKLVDSTDKADLRLIYLALAHMIKFRGH FLIEGDLNPX₂NSDVDKLFIQLVQTYNQLFEENPINASGVDAKAILSARLSKSRRLE NLIAQLPGEKKNGLFGNLIALSLGLTPNFKSNFDLAEDAKLQLSKDTYDDDLDNLL AQIGDQYADLFLAAKNLSDAILLSDILRVNTEITKAPLSASMIKRYDEHHQDLTLLK ALVRQQLPEKYKEIFFDQSKNGYAGYIDGGASQEEFYKFIKPILX₃KMDGTEELLV KLNX₄EDLLRKQRTFDNGSIPHQX₅HLGELHAILRRQEDFYPFLKDNREKIEKILTFR IPYYVGPLARGNSRFAWMTRKSEETITPWNFEEVVDKGASAQSFIERMTNFDKNLP NEKVLPKHX₆LLYEYFX₇VYNELTKVKYVTEGMRKPXsX₉LSGEQKKAIVDLLFKT NRKVTVKQLKEDYFKKIECFDSVEISGVEDRFNASLGTYHDLLKIIKDKDFLDNEE NEDILEDIVLTLTLFEDREMIEERLKTYAHLFDDKVMKQLKRRRYTGWGRLSRKLI NGIRDKQSGKTILDFLKSDGFANRX₁₀FMQLIHDDSLTFKEDIQKAQVSGQGDSLHE HIANLAGSPAIKX₁₁GILX₁₂TVKX₁₃VDELVKVMGRHKPEX₁₄IVIEMARENQTTQKG QKNSRERMKRIEEGIKELGSQILKEHPVENTQLQNEKLYLYYLQNGRDMYVDQEL DINRLSDYDVDHIVPQSFLKDDSIDNKVLTRSDKNRGKSDNVX₁₅SEEVVKKMKNY WRQLLNAKLITQRKFDNLTKAERGGLSELDKAGFIKRQLVETRQITKHVAQILDSR MNTKYDENDKLIREVKVITLKSKLVSDFRKDFQFYKVREINNYHHAHDAYLNAV VGTALIKKYPKLESEFVYGDYKVYDVRKMIAKSEQEIGKATAKYFFYSNIMNFFK TEITLANGEIRKRPLIETNGETGEIVWDKGRDFATVRKVLSMPQVNIVKKTEVQTG GFSKESILPKRNSDKLIARKKDWDPX₁₆KYGGFDSPTVAYSVLVVAKVEKGKSKKL KSVKELLGITIMERSSFEKNPIDFLEAKGYKEVKKDLIIKLPKYSLFELENGRKRML ASAGELQKGNELALPSX₁₇YVNFLYLASHYEKLKGSPEDNEQKX₁₈LFVEQHKHYL DEIIEQISEFSKRVILX₁₉DANLDKVLSAYNKHRDKPIREQAENIIHLFTLTNLGAPAA FKYFDTTIDRKRYTSTKEVLDATLIHQSITGLYETX₂₀IDLSQLGGD (SEQ ID NO: 2), wherein: X₁ comprises any amino acid except Leucine; X₂ comprises any amino acid except Aspartic acid; X₃ comprises any amino acid except Glutamic acid; X₄ comprises any amino acid except Arginine; X₅ comprises any amino acid except Isoleucine; X₆ comprises any amino acid except Serine; X₇ comprises any amino acid except Threonine; X₈ comprises any amino acid except Alanine; X₉ comprises any amino acid except Phenylalanine; X₁₀ comprises any amino acid except Asparagine; X₁₁ comprises any amino acid except Lysine; X₁₂ comprises any amino acid except Glutamine; X₁₃ comprises any amino acid except Valine; X₁₄ comprises any amino acid except Asparagine; X₁₅ comprises any amino acid except Proline; X₁₆ comprises any amino acid except Lysine; X₁₇ comprises any amino acid except Lysine; X₁₈ comprises any amino acid except Glutamine; X₁₉ comprises any amino acid except Alanine; X₂₀ comprises any amino acid except Arginine; or any combination thereof.

According to some embodiments, there is provided a Cas variant protein of a reference Cas protein comprising the amino acid sequence: MDKKYSIGX₁DIGTNSVGWAVITDEYKVPSKKFKVLGNTDRHSIKKNLIGALLFDS GETAEATRLKRTARRRYTRRKNRICYLQEIFSNEMAKVDDSFFHRLEESFLVEEDK KHERHPIFGNIVDEVAYHEKYPTIYHLRKKLVDSTDKADLRLIYLALAHMIKFRGH FLIEGDLNPDNSDVDKLFIQLVQTYNQLFEENPINASGVDAKAILSARLSKSRRLEN LIAQLPGEKKNGLFGNLIALSLGLTPNFKSNFDLAEDAKLQLSKDTYDDDLDNLLA QIGDQYADLFLAAKNLSDAILLSDILRVNTEITKAPLSASMIKRYDEHHQDLTLLKA LVRQQLPEKYKEIFFDQSKNGYAGYIDGGASQEEFYKFIKPILEKMDGTEELLVKL NREDLLRKQRTFDNGSIPHQIHLGELHAILRRQEDFYPFLKDNREKIEKILTFRIPYY VGPLARGNSRFAWMTRKSEETITPWNFEEVVDKGASAQSFIERMTNFDKNLPNEK VLPKHSLLYEYFTVYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 3), wherein: X₁ comprises any amino acid except Leucine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPX₁NSDVDKLFIQLVQTYNQLFEENPINASGVDAKAILSARLSKSRRLEN LIAQLPGEKKNGLFGNLIALSLGLTPNFKSNFDLAEDAKLQLSKDTYDDDLDNLLA QIGDQYADLFLAAKNLSDAILLSDILRVNTEITKAPLSASMIKRYDEHHQDLTLLKA LVRQQLPEKYKEIFFDQSKNGYAGYIDGGASQEEFYKFIKPILEKMDGTEELLVKL NREDLLRKQRTFDNGSIPHQIHLGELHAILRRQEDFYPFLKDNREKIEKILTFRIPYY VGPLARGNSRFAWMTRKSEETITPWNFEEVVDKGASAQSFIERMTNFDKNLPNEK VLPKHSLLYEYFTVYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 4), wherein: X₁ comprises any amino acid except Aspartic acid.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILX₁KMDGTEELLVKL NREDLLRKQRTFDNGSIPHQIHLGELHAILRRQEDFYPFLKDNREKIEKILTFRIPYY VGPLARGNSRFAWMTRKSEETITPWNFEEVVDKGASAQSFIERMTNFDKNLPNEK VLPKHSLLYEYFTVYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 5), wherein: X₁ comprises any amino acid except Glutamic acid.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN X₁EDLLRKQRTFDNGSIPHQIHLGELHAILRRQEDFYPFLKDNREKIEKILTFRIPYY VGPLARGNSRFAWMTRKSEETITPWNFEEVVDKGASAQSFIERMTNFDKNLPNEK VLPKHSLLYEYFTVYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 6), wherein: X₁ comprises any amino acid except Arginine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQX₁HLGELHAILRRQEDFYPFLKDNREKIEKILTFRIPYY VGPLARGNSRFAWMTRKSEETITPWNFEEVVDKGASAQSFIERMTNFDKNLPNEK VLPKHSLLYEYFTVYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 7), wherein: X₁ comprises any amino acid except Isoleucine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHX₁LLYEYFTVYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 8), wherein: X₁ comprises any amino acid except Serine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFX₁VYNELTKVKYVTEGMRKPAFLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 9), wherein: X₁ comprises any amino acid except Threonine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPX₁FLSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 10), wherein: X₁ comprises any amino acid except Alanine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAX₁LSGEQKKAIVDLLFKTNRKVTV KQLKEDYFKKIECFDSVEISGVEDRFNASLGTYHDLLKIIKDKDFLDNEENEDILEDI VLTLTLFEDREMIEERLKTYAHLFDDKVMKQLKRRRYTGWGRLSRKLINGIRDKQ SGKTILDFLKSDGFANRNFMQLIHDDSLTFKEDIQKAQVSGQGDSLHEHIANLAGS PAIKKGILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 11), wherein: X₁ comprises any amino acid except Phenylalanine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRX₁FMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 12), wherein: X₁ comprises any amino acid except Asparagine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKX₁GILQTVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 13), wherein: X₁ comprises any amino acid except Lysine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILX₁TVKVVDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 14), wherein: X₁ comprises any amino acid except Glutamine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKX₁VDELVKVMGRHKPENIVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 15), wherein: X₁ comprises any amino acid except Valine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPEX₁IVIEMARENQTTQKGQKNSRERMKRIE EGIKELGSQILKEHPVENTQLQNEKLYLYYLQNGRDMYVDQELDINRLSDYDVDH IVPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 16), wherein: X₁ comprises any amino acid except Asparagine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVX₁SEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 17), wherein: X₁ comprises any amino acid except Proline.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPX₁KYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETRIDLSQLGGD (SEQ ID NO: 18), wherein: X₁ comprises any amino acid except Lysine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SX₁YVNFLYLASHYEKLKGSPEDNEQKQLFVEQHKHYLDEIIEQISEFSKRVILADA NLDKVLSAYNKHRDKPIREQAENIIHLFTLTNLGAPAAFKYFDTTIDRKRYTSTKE VLDATLIHQSITGLYETRIDLSQLGGD (SEQ ID NO: 19), wherein: X₁ comprises any amino acid except Lysine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKX₁LFVEQHKHYLDEIIEQISEFSKRVILADA NLDKVLSAYNKHRDKPIREQAENIIHLFTLTNLGAPAAFKYFDTTIDRKRYTSTKE VLDATLIHQSITGLYETRIDLSQLGGD (SEQ ID NO: 20), wherein: X₁ comprises any amino acid except Glutamine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILX₁DA NLDKVLSAYNKHRDKPIREQAENIIHLFTLTNLGAPAAFKYFDTTIDRKRYTSTKE VLDATLIHQSITGLYETRIDLSQLGGD (SEQ ID NO: 21), wherein: X₁ comprises any amino acid except Alanine.

According to some embodiments, there is provided a Cas variant protein of a reference Cas protein comprising the amino acid sequence: MDKKYSIGLDIGTNSVGWAVITDEYKVPSKKFKVLGNTDRHSIKKNLIGALLFDSG ETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHF LIEGDLNPDNSDVDKLFIQLVQTYNQLFEENPINASGVDAKAILSARLSKSRRLENL IAQLPGEKKNGLFGNLIALSLGLTPNFKSNFDLAEDAKLQLSKDTYDDDLDNLLAQ IGDQYADLFLAAKNLSDAILLSDILRVNTEITKAPLSASMIKRYDEHHQDLTLLKAL VRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYV GPLARGNSRFAWMTRKSEETITPWNFEEVVDKGASAQSFIERMTNFDKNLPNEKV LPKHSLLYEYFTVYNELTKVKYVTEGMRKPAFLSGEQKKAIVDLLFKTNRKVTVK QLKEDYFKKIECFDSVEISGVEDRFNASLGTYHDLLKIIKDKDFLDNEENEDILEDIV LTLTLFEDREMIEERLKTYAHLFDDKVMKQLKRRRYTGWGRLSRKLINGIRDKQS GKTILDFLKSDGFANRNFMQLIHDDSLTFKEDIQKAQVSGQGDSLHEHIANLAGSP AIKKGILQTVKVVDELVKVMGRHKPENIVIEMARENQTTQKGQKNSRERMKRIEE GIKELGSQILKEHPVENTQLQNEKLYLYYLQNGRDMYVDQELDINRLSDYDVDHI VPQSFLKDDSIDNKVLTRSDKNRGKSDNVPSEEVVKKMKNYWRQLLNAKLITQR KFDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLES EFVYGDYKVYDVRKMIAKSEQEIGKATAKYFFYSNIMNFFKTEITLANGEIRKRPLI ETNGETGEIVWDKGRDFATVRKVLSMPQVNIVKKTEVQTGGFSKESILPKRNSDK LIARKKDWDPKKYGGFDSPTVAYSVLVVAKVEKGKSKKLKSVKELLGITIMERSS FEKNPIDFLEAKGYKEVKKDLIIKLPKYSLFELENGRKRMLASAGELQKGNELALP SKYVNFLYLASHYEKLKGSPEDNEQKQLFVEQHKHYLDEIIEQISEFSKRVILADAN LDKVLSAYNKHRDKPIREQAENIIHLFTLTNLGAPAAFKYFDTTIDRKRYTSTKEVL DATLIHQSITGLYETX₁IDLSQLGGD (SEQ ID NO: 22), wherein: X₁ comprises any amino acid except Arginine.

In some embodiments, the reference Cas protein comprises a Streptococcus pyogenes Cas wildtype (SpyCas) protein.

In some embodiments, the reference is selected from: eSpCas9(1.1), SpCas9-HF1, HypaCas9, evoCas9, Sniper-Cas9, Hifi-Cas9, or LZ3 Cas9.

In some embodiments, the reference is eSpCas9(1.1).

In some embodiments, there is provided an eSpCas9(1.1) variant comprising at least one amino acid substitution in position selected from: K848, K1003, R1060, or any combination thereof. In some embodiments, the eSpCas9(1.1) variant comprises at least one amino acid substitution in position selected from: K848A, K1003A, R1060A, or any combination thereof.

In some embodiments, there is provided a HiFiCas9 variant comprising an amino acid substitution in position R691. In some embodiments, the HiFi-Cas9 variant comprises the amino acid substitution R691A.

In some embodiments, there is provided a HypaCas9 variant comprising at least one amino acid substitution in position selected from: N692, M694, Q695, H698 or any combination thereof. In some embodiments, the HypaCas9 variant comprises at least one amino acid substitution in position selected from: N692A, M694A, Q695A, H698A, or any combination thereof.

In some embodiments, a HypaCas9 variant of the invention further comprises one or more amino acid substitution(s) compared to a wildtype Cas9. In some embodiments, the one or more amino acid substitution(s) compared to the wildtype Cas9 comprise or consist of the amino acid substitution(s) disclosed herein.

In some embodiments, there is provided a SniperCas9 variant comprising at least one amino acid substitution in position selected from: F539, M763, K890 or any combination thereof. In some embodiments, the SniperCas9 variant comprises at least one amino acid substitution in position selected from: F539S, M763I, K890N, or any combination thereof.

In some embodiments, there is provided a HF1-Cas9 variant comprising at least one amino acid substitution in position selected from: N497, R661, Q695, Q926 or any combination thereof. In some embodiments, the HF-1Cas9 variant comprises at least one amino acid substitution in position selected from: N974A, R661A, Q695A, Q926A, or any combination thereof.

In some embodiments, there is provided an evoCas9 variant comprising at least one amino acid substitution in position selected from: M495, Y515, K526, R661 or any combination thereof. In some embodiments, the evoCas9 variant comprises at least one amino acid substitution in position selected from: M495V, Y515N, K526E, R661L, or any combination thereof.

In some embodiments, there is provided a LZ3 Cas9 variant comprising at least one amino acid substitution in position selected from: N690, T691, G915, N980 or any combination thereof. In some embodiments, the LZ3 Cas9 variant comprises at least one amino acid substitution in position selected from: N690C, T691I, G915M, N980K, or any combination thereof.

In some embodiments, the Cas variant protein comprises or is characterized by having improved function, compared to the Cas reference protein.

In some embodiments, the improved function is selected from: catalytic activity, specificity, stability, or any combination thereof. In some embodiments, stability comprises or is thermostability.

In some embodiments, the improved function is thermostability.

In some embodiments, the at least one amino acid substitution is selected from: N692V, K1129T, K1231I, L9R, T519V, D177A, E381N, R395A, I414A, S512R, A538I, F539V, K735W, Q739R, V743N, N758V, P871I, Q1256W, A1283E, R1359Q, or any combination thereof.

In some embodiments, the at least one amino acid substitution is selected from: N692V, K1129, T, K1231I, L9R, T519V, or any combination thereof.

In some embodiments, the at least one amino acid substitution is of N692V.

In some embodiments, the at least one amino acid substitution is N692V.

According to some embodiments, there is provided a nucleic acid molecule comprising a nucleic acid sequence encoding the Cas variant protein disclosed herein.

According to some embodiments, there is provided a vector comprising the nucleic acid sequence disclosed herein. In some embodiments, the vector is an expression vector. In some embodiments, the vector is a plasmid.

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

A person with skill in the art will appreciate that a gene can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

According to some embodiments, there is provided a cell comprising any one of: (a) the Cas variant protein disclosed herein; (b) the nucleic acid sequence disclosed herein; (c) the vector disclosed herein; or (d) any combination of (a) to (c).

In some embodiments, the cell is a recombinant cell. In some embodiments, the cell is a transgenic cell. In some embodiments, the cell is a transformed cell. In some embodiments, the cell is a host cell.

In some embodiments, the cell is a cell of a unicellular organism. In some embodiments, the cell is a bacterial cell or a fungus. In some embodiments, the cell is a cell of a multicellular organism.

In some embodiments, the cell is a mammalian cell or a human cell.

According to some embodiments, there is provided a composition comprising any one of: (a) the Cas variant protein disclosed herein; (b) the nucleic acid sequence disclosed herein; (c) the vector disclosed herein; (d) the cell disclosed herein; or (e) any combination of (a) to (d), and an acceptable carrier, diluent, excipient, or adjuvant.

In some embodiments, the carrier is a pharmaceutically acceptable carrier.

In some embodiments, the composition is a pharmaceutical composition.

As used herein, the terms “carrier”, “excipient”, or “adjuvant” refer to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Use

According to some embodiments, there is provided a method for modifying at least one target nucleic acid sequence of interest in a cell.

According to some embodiments, there is provided a method of treating, preventing, reducing, delaying the onset, or ameliorating a pathologic disorder in a subject in need thereof.

In some embodiments, the method comprises administering to the subject a therapeutically effective amount of: (a) any one of: (i) the Cas variant protein disclosed herein; (ii) the nucleic acid sequence disclosed herein; (iii) the expression vector disclosed herein; and (iv) any combination of (i) to (iii); and (b) at least one target recognition element or a nucleic acid sequence encoding thereof.

In some embodiments, the method comprises contacting a cell with an effective amount of: (a) any one of: (i) the Cas variant protein disclosed herein; (ii) the nucleic acid sequence disclosed herein; (iii) the expression vector disclosed herein; and (iv) any combination of (i) to (iii); and (b) at least one target recognition element or a nucleic acid sequence encoding thereof.

In some embodiments, the target nucleic acid sequence is associated with at least one pathologic disease or disorder.

In some embodiments, the pathological disorder is selected from: proliferative disorder, a congenital disorder, an immune-related condition, an inflammatory condition, a metabolic disorder, a disorder caused by a pathogen, an autoimmune disorder, a disorder associated with the expression of a coding or non-coding sequence, an inborn error of metabolism (IEM) disorder, or any combination thereof.

In some embodiments, the pathological disorder is induced by, results from, propagated due to, characterized by, or any combination thereof, of a loss of function mutation in an encoding gene.

As used herein, the term “loss of function mutation” encompasses any one of non-synonymous and nonsense mutation, rendering a protein product of the encoding gene less functional, dysfunctional, non-functional, abnormally functional, or any combination thereof.

In some embodiments, a loss of function mutation induces or leads to a premature stop codon.

In some embodiments the cell comprises cell of a subject. In some embodiments, the cell comprises a cell obtained or derived from a subject.

In some embodiments, the cell or a composition comprising same, is suitable for use in allogeneic or autologous transplantation.

In some embodiments, the cell is from an allogeneic source or an autologous source.

In some embodiments, contacting comprises administering to the subject.

In some embodiments, administering comprises administering a therapeutically effective amount of (a) any one of: (i) the Cas variant protein disclosed herein; (ii) the nucleic acid sequence disclosed herein; (iii) the expression vector disclosed herein; and (iv) any combination of (i) to (iii); and (b) at least one target recognition element or a nucleic acid sequence encoding thereof, to the subject.

The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

According to some embodiments, there is provided a therapeutic combination comprising: (a) at least one of: Cas variant protein disclosed herein, the nucleic acid molecule disclosed herein, the cell disclosed herein, or the pharmaceutical composition disclosed herein; (b) at least one target recognition element or any nucleic acid sequence encoding thereof. In some embodiments, combination is for use in a method of modifying at least one target nucleic acid sequence of interest in at least one cell.

According to some embodiments, there is provided a therapeutic combination comprising: (a) at least one of: Cas variant protein disclosed herein, the nucleic acid molecule disclosed herein, the cell disclosed herein, or the pharmaceutical composition disclosed herein; (b) at least one target recognition element or any nucleic acid sequence encoding thereof; and (c) at least one composition comprising at least one of (a) and (b). In some embodiments, the combination is for use in a method of treating, preventing, reducing, delaying the onset, or ameliorating a pathologic disorder in a subject in need thereof.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, “treating” comprises ameliorating and/or preventing.

In some embodiments, ameliorating comprises alleviating at least one symptom associated with a disease as described herein.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### In Silico Analysis

The structure of the SpyCas9 complex was taken from the Protein Data Bank (PDB-101; accession numbers PDB: 5F9R). Next, using the mutagenesis plugin in PyMol Molecular Graphics System Version 1.8 (Schrödinger, LLC., Cambridge, MA), the inventors performed in silico bases mutagenesis of the given gRNA (chain A) and DNA (chain C—TS-DNA and chain D—NTS-DNA) to the gRNA and DNA sequences used in the study of Hsu et al. This in silico mutagenesis of bases was made with X3dna-DSSR (https://x3dna.org/) Linux package. For the WT structure now modified with four new gRNA sequences, the inventors created a structure for each of the possible mismatches in positions 1-19. WT and mismatched structures were analyzed by an ENCoM coarse-grained NMA method to evaluate the effect of the analyzed mismatch on the stability of the protein and the DNA. This method is based on an entropic considerations C package of ENCoM available at the ENCoM development website (https://github.com/NRGlab/ENCoM), compiled and used on a Ubuntu platform (Canonical Group, UK). For each analyzed variant, the inventors calculated the entropy difference (ΔG) by subtracting the NMA-based mismatched structure's entropic profile from the entropic profile of the WT perfect-match structure model.

The calculation of the entropic difference (ΔG) was performed using MATLAB software (MathWorks, Natick, MA).

Next, to build the nine high fidelity structures, the Mutagenesis plugin in PyMol Molecular Graphics System Version 1.8 (Schrödinger, LLC., Cambridge, MA) was used to perform the appropriate in silico point mutagenesis in the WT with changed gRNA modelled structure (EMX1 site 3). Using this structure, in silico mutagenesis was performed for each variant to replace the amino acids in accordance with each of the eight variants. These in silico mutations were made only in chain B. All variants were also analyzed for mismatches in the gRNA by the same procedure as described above. Mismatched structures of all variants were analyzed by an ENCoM coarse-grained NMA method to evaluate the effect of the analyzed mismatch on the stability of the protein. For each analyzed variant, the inventors calculated the ΔG by subtracting the NMA-based mismatched structure's entropic profile from the entropic profile of the perfect-match structure model. The calculation of the ΔG was done using MATLAB software.

### Screening and Selection of Active and High-Fidelity Variants

In order to predict the activity of a newly suggested variant, the inventors first fit linearly the known empirical results of known variants with their ΔG score, thus, obtaining the following linear function:

Activity=aΔG+b.

By substituting the in silico ΔG value of a new candidate variant, that still does not have empirical data, the inventors obtain the predicted activity. In order to predict the specificity of a newly suggested variant, the inventors first fit linearly the known sorted empirical results of known variants with the gradient of ΔG score of known variants, thus, obtaining the following linear function:

Specificity=a(ΔΔG)+b.

The inventors added the in silico ΔG value of a new candidate variant, each time in a different position between the known variants ΔG score and taking the gradient. The ΔΔG resulting with the best linear fit, is the ΔΔG of choice. Upon calculating the ΔΔG of a new variant, the inventors substitute it into the equation above with the resultant expected specificity. Using the functions described herein, the variants that were generated by the diversification process, were selected for being both: (a) as active as wildtype Cas9 (reference enzyme); and (b) more specific than wildtype Cas9 and as specific as known high-fidelity variants.

### NMA, Entropy Calculations and Structure Alterations

The structure of the SpyCas9 complex was taken from the Protein Data Bank (PDB-101; accession numbers PDB: 5F9R). Next, using the mutagenesis plugin in PyMol Molecular Graphics System Version 1.8 (Schrödinger, LLC., Cambridge, MA), the inventors performed in silico bases mutagenesis of the given gRNA (chain A) and DNA (chain C—TS-DNA and chain D—NTS-DNA) to the gRNA and DNA sequences used in the study of Hsu et al. This in silico mutagenesis of bases was made with X3dna-DSSR (https://x3dna.org/) Linux package. For the WT structure now modified with four new gRNA sequences, the inventors created a structure for each of the possible mismatches in positions 1-19. WT and mismatched structures were analyzed by an ENCoM coarse-grained NMA method to evaluate the effect of the analyzed mismatch on the stability of the protein and the DNA. This method is based on an entropic considerations C package of ENCoM available at the ENCoM development website (github.com/NRGlab/ENCoM), compiled and used on a Ubuntu platform (Canonical Group, UK). For each analyzed variant, the inventors calculated the entropy difference (ΔG) by subtracting the NMA-based mismatched structure's entropic profile from the entropic profile of the WT perfect-match structure model.

The calculation of the entropic difference (ΔG) was done using MATLAB software (MathWorks, Natick, MA).

Next, to build the nine high fidelity structures, the Mutagenesis plugin in PyMol Molecular Graphics System Version 1.8 (Schrödinger, LLC., Cambridge, MA) was used to perform the appropriate in silico point mutagenesis in the WT with changed gRNA modelled structure (EMX1 site 3). Using this structure, in silico mutagenesis was performed for each variant to replace the amino acids in accordance with each of the eight variants. These in silico mutations were made only in chain B. All variants were also analyzed for mismatches in the gRNA by the same procedure as described above. Mismatched structures of all variants were analyzed by an ENCoM coarse-grained NMA method to evaluate the effect of the analyzed mismatch on the stability of the protein. For each analyzed variant, the inventors calculated the ΔG by subtracting the NMA-based mismatched structure's entropic profile from the entropic profile of the perfect-match structure model. The calculation of the ΔG was done using MATLAB software.

### Cell Culture, Transduction and Transfection

HEK293 ft cells were grown in standard media (DMEM high glucose, L-glu, Gibco 41965039; 10% FBS, Gibco 10270-106; 1% PS, Gibco 15070063) at 37° C. with 5% CO₂. To establish the EGFP-PEST stable cell line the inventors used a lentivirus carrying the gene of interest (EGFP-PEST-P2A-PuroR). After transduction, infected cells were grown under selection with puromycin (Thermo-Fisher J67236, 1:1000) for two weeks. For all experiments, cells where transfected with Lipofectamine3000 (Invitrogen L3000015) according to the manufacturer's protocol, with Opti-Mem (Gibco 31985-047). All transfections were carried in 96-well plates. 8×10³ cells/well were seeded on day 0. Total of 40 ng plasmid DNA/well were used for transfection (sgRNA:Cas ratio 1:1, supplemented with mCherry reporter plasmid for transfection assessment and flow cytometry gating) with 0.2 μl/well Lipofectamine3000. Media replacement was performed 24 hr after transfection. The EMX1 and VEGFA site 3 gRNA sequences were cloned into BPK1520 according to the standard protocol. All other gRNAs and Cas enzymes were cloned into expression vectors by VectorBuilder.

### DNA Extraction

For rhAmpSeq analyses DNA was extracted 96 hours post transfection according to the following protocol, based on a previous publication with minor changes (Doman et al., (2020)). Media were removed, and cells were carefully washed with PBS. After washing, cells are lysed with 150 μl lysis buffer (10 mM Tris-HCl pH 8, Sigma T3038; 0.05% SDS, Bio-Basic SD8119; 35 μg/ml Proteinase K, Roche 03-115-879-001). After 1 hr incubation at 37° C., lysed cells were pipetted, and the solution was transferred to PCR tubes. Tubes were incubated at 80° C. using a thermocycler. The solution of extracted DNA was used as the DNA template for the NGS analysis.

rhAmpSeq Analysis

For each target (EMX1, VEGFA and AAVS1), a PCR primers panel was designed using IDT's rhAmpSeq designated tool. Library preparation and NGS were conducted according to IDT's protocol. CRISPResso2 was used to analyze NGS data and assess the on and off-target activity of the Cas variants for each gRNA (using the CRISPRessoPooled utility).

### GFP Disruption Assay

EGFP-PEST stable cells were co-transfected with one of the sgRNA plasmids and one of the Cas plasmids, together with a reporter plasmid. Five (5) days post transfection cells were trypsinized, incubated 5 min at 37° C., and neutralized by 150 μl FBS-enriched FACS buffer (PBS; 5% FBS; 25 mM HEPES Biological industries (Israel) 03-025; 5 mM EDTA, Biological industries (Israel) 01-862). The suspended cells were filtered using a mesh-capped plate (Merck MANMN4010) before flow cytometry analysis. Cells were analyzed by flow cytometry (CytoFLEX S, Beckman Coulter) to assess GFP-disruption rates. Decrease in GFP levels was assessed by measuring the GFP positive cells, based on GFP+ untreated cells, out of mCherry positive cells. The GFP disruption rate was calculated as following:

$$\% GFP disruption = 100 \times \frac{100 - \% {GFP}^{+} - \% background}{\% perfect match sgRNA}$$

Each plate contained its internal positive and negative controls for normalization.

### Statistical Tests

The GraphPad Prism v9.4.1 software was used to produce all charts and analyze data. Statistical tests were performed as described in the Figure legends and P values of ≤0.05 are labeled with a single asterisk (*), in contrast to P values of ≤0.01 (**), ≤0.001 (***), or ≤0.0001 (****); where not indicated, P values are non-significant. In all relevant Figure panels, values of mean±SEM are reported, and the exact ‘n’ value is described in each Figure legend.

### Example 1

### NMA Accurately Replicates Empiric Data of SpyCas9 Activity and Specificity Profile

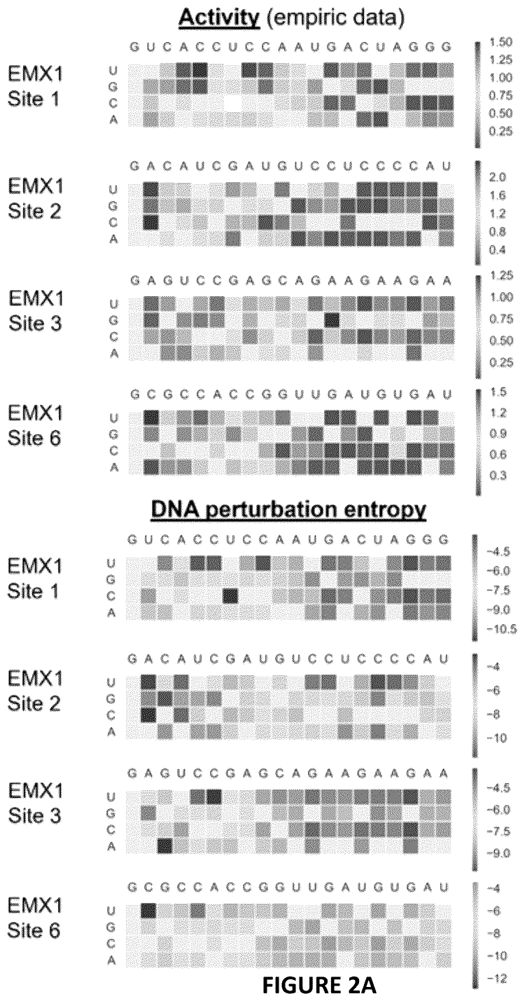

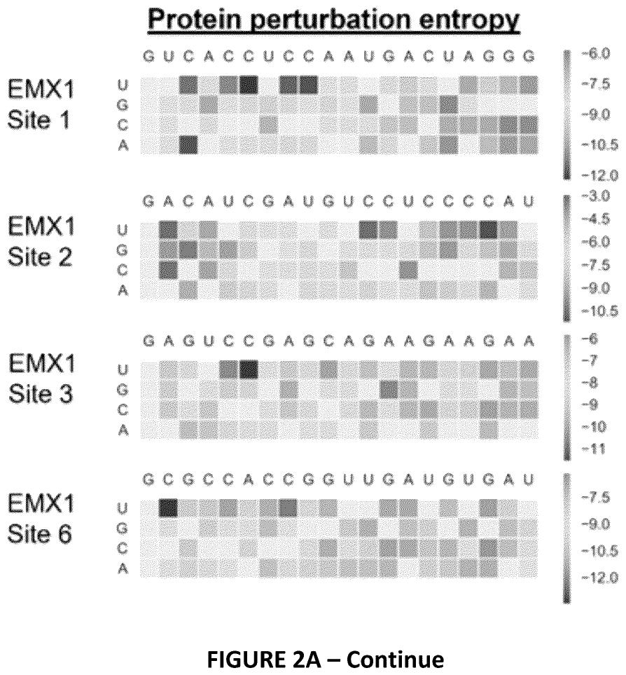

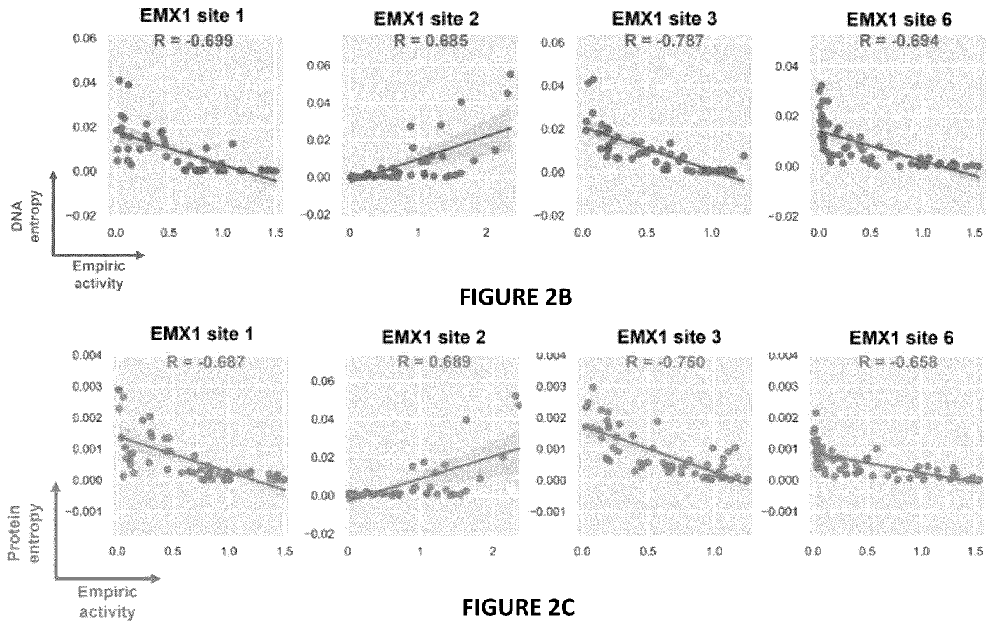

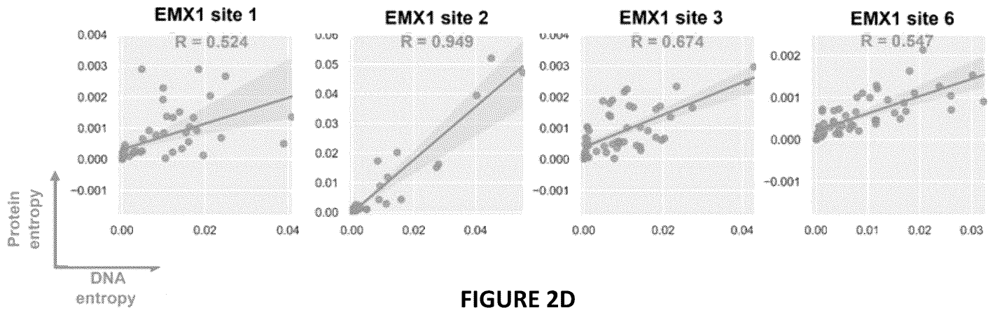

To test the relevance of NMA to predict the activity of SpyCas9, the inventors performed an in-silico replication of a previously published experiment by Hsu et al. The empiric data describe the specificity profile of SpyCas9 in four genomic loci within the EMX1 gene. The specificity was measured as the cleavage activity in the presence of mismatches between the single-guide RNA (sgRNA) and the target DNA, compared to a perfect-match sgRNA (FIG. 2A, left column). The inventors hypothesized that the entropic changes caused by single-nucleotide mismatches would reflect the specificity patterns obtained from the experimental results. To examine the current hypothesis, the structure of the SpyCas9 complex (bound to the sgRNA and the target DNA) was fetched from the Protein Data Bank (PDB, accession number: 5F9R) and the RNA and DNA sequences were modified to match the four EMX1 loci. The inventors then generated 57 modified structures per locus, where in each structure, one nucleotide of the sgRNA was changed, according to the original experiment (see Methods). In total, 232 structures were generated. The ΔG of each structure was measured using NMA. Since we have modified the sgRNA molecule in the structure, we sought to assess the single-nucleotide mismatch effect on the ΔG of the protein (chain B) and the DNA (chain C—target strand) separately (FIG. 2). The patterns unveiled from the ΔG measurements from both the protein and the DNA indeed resemble the empiric data at the different EMX1 sites (FIG. 2A). Moreover, the seed region can be observed clearly in the ΔG patterns, demonstrating the consistency of our results with the previous reports. The correlation for each combination of empiric results, ΔG of the protein and ΔG of the DNA, was calculated for all sites (FIGS. 2B-2D). The Pearson correlation coefficient (R) of the DNA entropy or the protein entropy with the empiric data is very similar and ranges from 0.6853 to 0.7875 (FIG. 2B) and 0.6577 to 0.7502 (FIG. 2C), respectively (absolute values). The high R values demonstrate the feasibility of in silico NMA to predict the activity outcome of SpyCas9, even when the DNA and sgRNA sequences of the structures are modified compared to the original structure. Since the |RI values of the EMX1 site 3 are higher compared to the three other sites, we decided to perform the following analyses in this study on the EMX1 site 3. R values and P-values are summarized in Table 1.

TABLE 1
Summary of Pearson's r and P-value

| | EMX1 loci | | | |
|---|---|---|---|---|
| N = 57 | Site 1 | Site 2 | Site 3 | Site 6 |
| DNA entropy | r = −0.699 | r = 0.685 | r = −0.787 | r = −0.694 |
| Empiric activity | P.V. < .00001 | | | |
| Protein entropy | r = −0.687 | r = 0.689 | r = −0.75 | r = −0.658 |
| Empiric activity | P.V. < .00001 | | | |
| DNA entropy | r = 0.524 | r = 0.949 | r = 0.674 | r = 0.547 |

| | | | |
|---|---|---|---|
| Protein entropy | P.V. = .000029 | P.V. < .00001 | P.V. = .000011 |

### Example 2

Mismatched gRNAs Targeting Different Loci Produce Consistent Correlation Patterns Between the Entropy of Residues and the Empiric Enzyme Activity

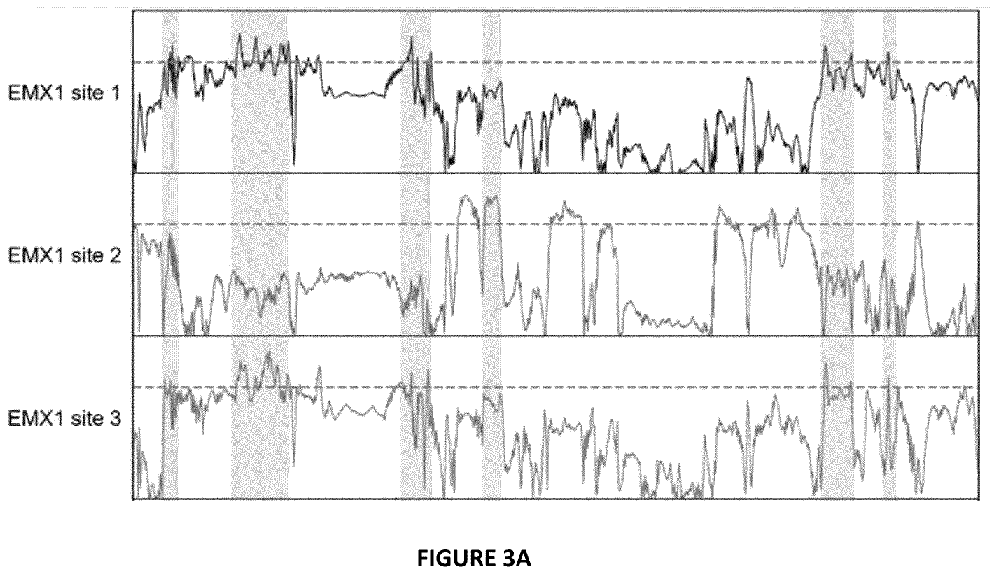

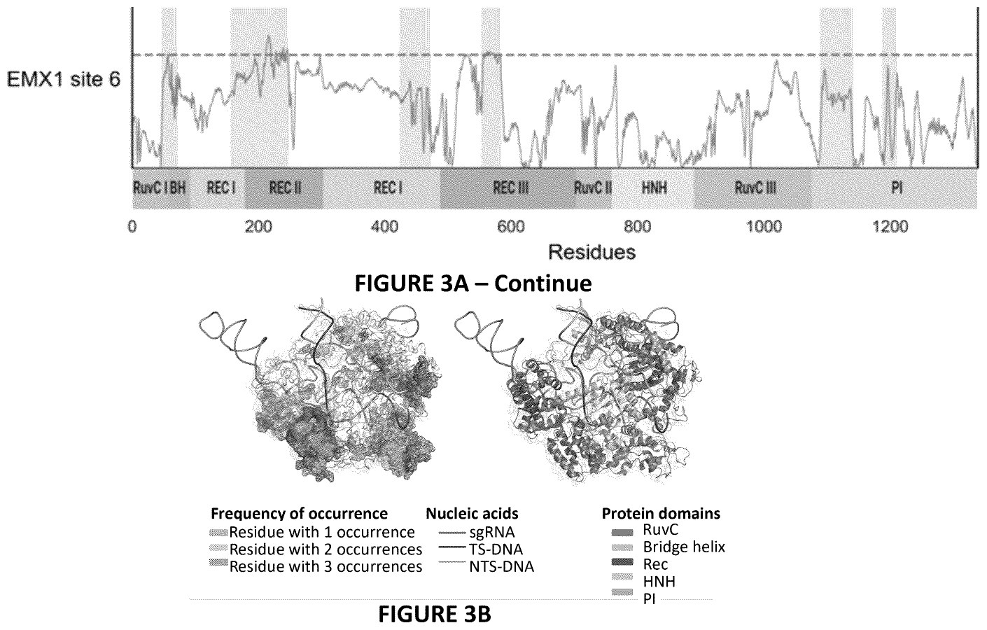

To examine which amino acids within the structure of SpyCas9 respond in the form of ΔG changes coordinately with activity rates in the presence of mismatches, the inventors checked the ΔG of each residue. Using the coarse-grained NMA method, the ΔG was calculated for the α-carbon of each residue. The correlation between the ΔG and the activity was calculated (R) and plotted for each genomic site (FIG. 3A). It is apparent that the R values for each amino acid are highly consistent among the four EMX1 loci, indicating the coherence reactivity of the protein regions in varying genetic contexts. Noticeably, high R values were most abundant within the REC lobe (REC domains I-III) and the PAM interacting (PI) domain, as well as the bridge-helix (BH) that is known to confer mismatch sensitivity. The inventors set a tentative threshold of R=0.55 and marked regions of residues that cross it in more than one EMX1 site, indicating protein regions where entropic response to mismatches harmoniously correlates with the empiric activity of the enzyme. Further to the 2D representation of the residues crossing the R=0.55 threshold, the inventors depicted the number of occurrences in which a residue crossed the threshold in a 3D representation to observe the structural relevance of such residues (FIG. 3B). Examination of the 3D structure confirms that residues that repeatedly have high R values are likely to interact directly with the nucleic acids within the structure. For instance, residues 164-174, which are part of the REC lobe (REC I domain), interact closely with the gRNA, stabilizing the R-loop (gRNA:TS-DNA heteroduplex), and cross the R threshold in two EMX1 sites. Remarkably, although the REC2 domain does not bind the gRNA and the DNA (despite residue D269), and SpyCas9 still retains its activity even after complete removal of the domain, it contains the most frequent residues (212-219 and 244-246).

### Example 3

### NMA-Based Predictions of the Activity and Specificity of Engineered SpyCas9 Variants

As a modification of nucleic acids within the structure of SpyCas9 led to NMA-based results that were consistent with empiric data, we speculated whether NMA might also predict the outcome of amino acids modifications. Similar to the comparison of ΔG to the activity in the presence of mismatches (FIG. 2), the computationally modified protein should be compared to a priori empiric data of such variants. To that end, the inventors obtained the specificity and activity scores of eight engineered SpyCas9 variants with improved specificity from a previously published study by Schmid-Burgk et al. This study provides high-throughput and uniformly collected data (using the TTISS method) of all eight variants, compared to the wildtype (WT) SpyCas9. The variants that were compared were eSpCas9(1.1), SpCas9-HF1, HypaCas9, evoCas9, Sniper-Cas9, Hifi-Cas9, and LZ3 Cas9. The authors tested 59 gRNAs to evaluate the on-target activity and specificity (genome-wide off-target activity), thus, generating comprehensive and robust data.

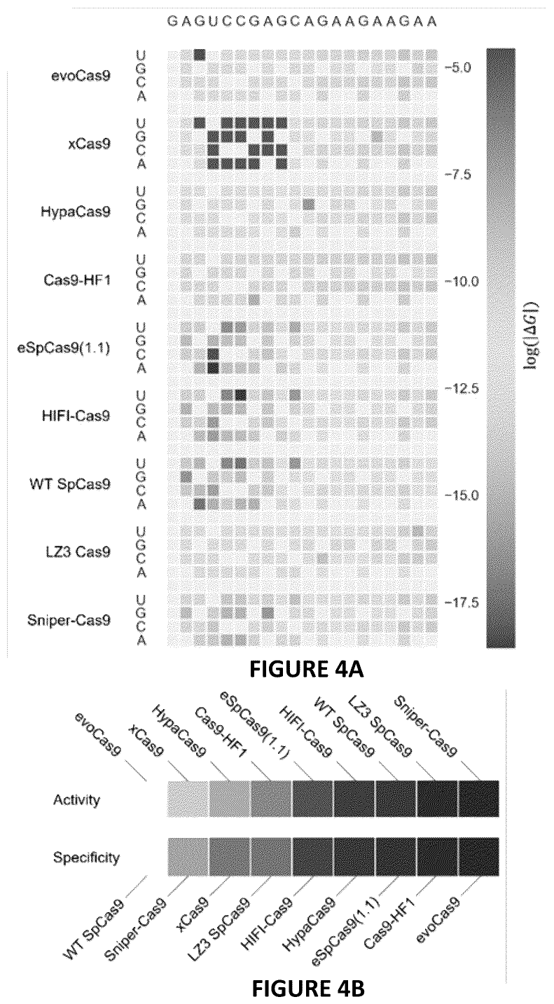

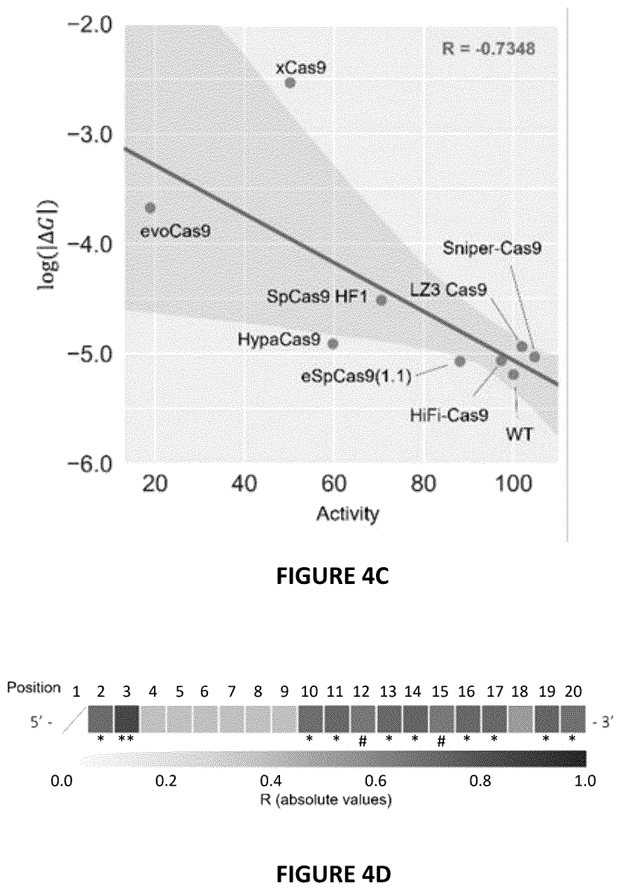

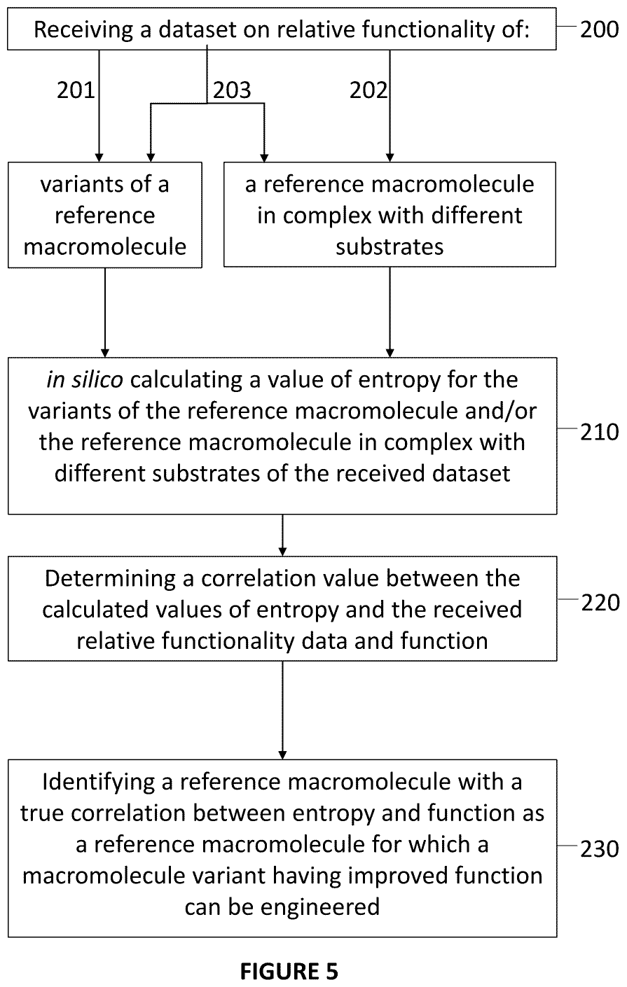

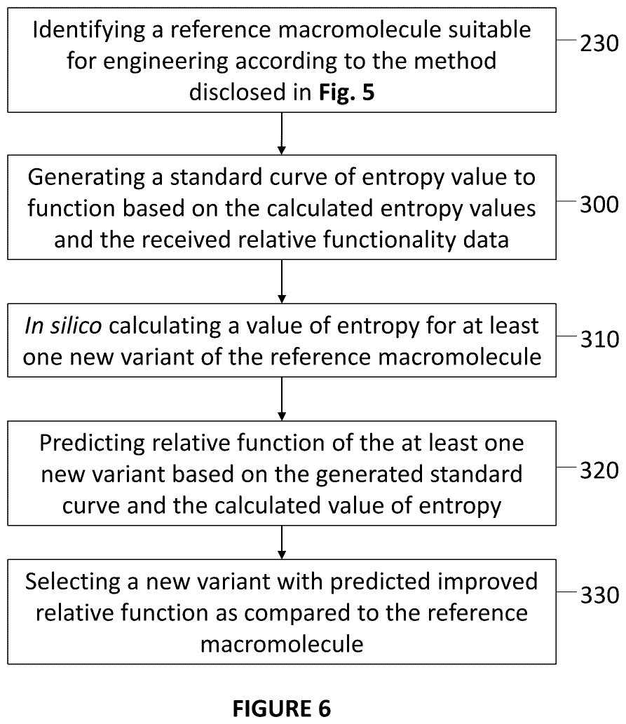

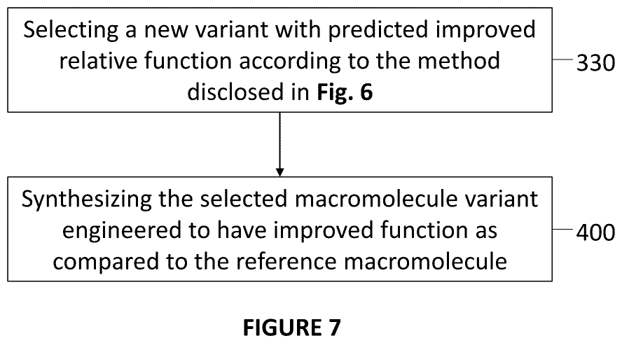

The inventors focused on the protein structure with the altered nucleic acids corresponding to the EMX1 site 3 sequence and modified the amino acids according to the various engineered SpyCas9 variants. Thereafter, by generating structures of all the single mismatches for each variant (as previously described herein), the inventors established a predicted specificity profile consisting of SpyCas9 and eight variants (FIG. 4A). The order of the variants was determined according to their activity, as measured by Schmid-Burgk and colleagues (FIG. 4B). The two most specific variants, evoCas9 and Cas9-HF1, exhibited highly specific entropy profiles compared to WT SpyCas9 and other less specific variants. Interestingly, the ΔG values are highly correlative with the average on-target activity scores (R=−0.7348; FIG. 4C). While most variants show ΔG patterns that correlate with the empiric activity, xCas9 was seemingly not in line with the other variants. xCas9 was comprised of seven mutations and was initially screened as a PAM-modified variant that afterwards was found to have improved specificity. The inconsistent entropy pattern may be due to other molecular mechanisms underlying the specificity improvement and activity reduction of xCas9. The inventors next calculated the correlation between the average ΔG of each position and each variant and the average activity score of each variant (FIG. 4D). High R values indicate the feasibility to predict the activity outcome based on the ΔG of a particular position. Surprisingly, the obtained R values pattern in the different positions of the gRNA resembles the seed region pattern, excluding positions two and three (PAM-distant region) that are thought to be the least stringent. These significantly correlative positions (2, 3, 10-17, 19 and 20) can be of great use in predicting the on-target activity of various Cas variants and serve as predictors for off-targets assessments.

### Discussion

The data presented in this study demonstrate the correlation between NMA and empiric enzymatic activity from experimental studies. The multicomponent complex of Cas9 protein, sgRNA and DNA (TS and NTS-DNA) allowed the inventors to manipulate one or two elements (gRNA mismatches or protein mutations) and measure their influence on the constants (i.e., DNA). Strong correlations were observed after changes were made to the original structure. Strikingly, after also changing the protein residues the correlation remained as strong. While examining different hypotheses, whether NMA correlates with WT SpyCas9 in the presence of mismatches and if SpyCas9 variants correlate with their reported activity, the inventors utilized two independent datasets. One, by Hsu et al., characterizes the specificity profile of WT SpyCas9 in four loci within the EMX1 gene. The other, by Schmid-Burgk et al., compares eight variants with improved specificity and attempts to find genome-wide off-targets and determine their on-target efficiency. The consistent high correlation between NMA and empiric experimental data from different studies provide strong evidence for the validity of NMA to predict the outcome of Cas9 activity. The principle presented herein may lay the groundwork for future tools and technologies such as off-targets assessment tools and engineering of novel Cas variants. The latter can be particularly benefited from the NMA activity-based standard curve (FIG. 4) or a similar NMA specificity-based curve. Moreover, similar analysis of other Cas enzymes (Cas9, Cas12 or other effector proteins) can bring to novel effector proteins from different classes with unique functions. This is restraint to the limitations of the method, as it requires available structure of the protein of interest in association with related molecules (e.g., DNA, RNA), and detailed data that can be used for comparison and calibration. Any engineered protein candidate that was predicted using this method should be tested experimentally in a “wet lab”. Notably, actual experimental results may be subjected to variance resulted from multiple parameters. This may affect both the empiric data used for analysis and the validation experiment of the proteins of interest. Furthermore, structures depicting the protein (or complex) in different conformations might result in different conclusions. Taken together, this study emphasizes the feasibility and accuracy of NMA in the context of the gene editing by the CRISPR-Cas9 system. The findings described herein may have broad implications on future novel Cas9 variants and specificity assessment tools.

### Example 4

### Improved Macromolecule Variant(s)—In Vitro Analysis

Normal mode analysis (NMA)-based in silico directed evolution was applied on SpyCas9 to identify novel variants with improved specificity. Out of the 19 predicted candidates, 4 were tested and compared to wildtype Cas9 as well as two known high fidelity (HF) variants: eSpCas9(1.1) and HiFi-Cas9, in vitro.

Plasmids containing the candidates were constructed and transfected into HEK293FT cells. Genomic DNA was analyzed using rhAmpSeq according to previous data (based on GUIDE-seq; Schmid-Burgk et al., Mol. Cell, 2020).

Briefly, rhAmpSeq is an NGS-based method to identify Cas9 activity in its on-target site (i.e., activity) and known off-target sites (i.e., specificity). HF variants were expected to achieve satisfying on-target editing levels, while maintaining low as possible off-target editing levels.

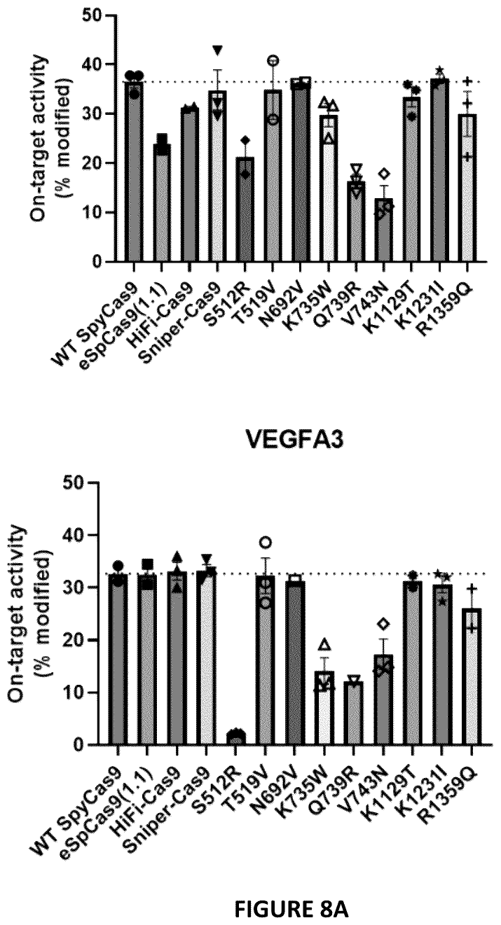

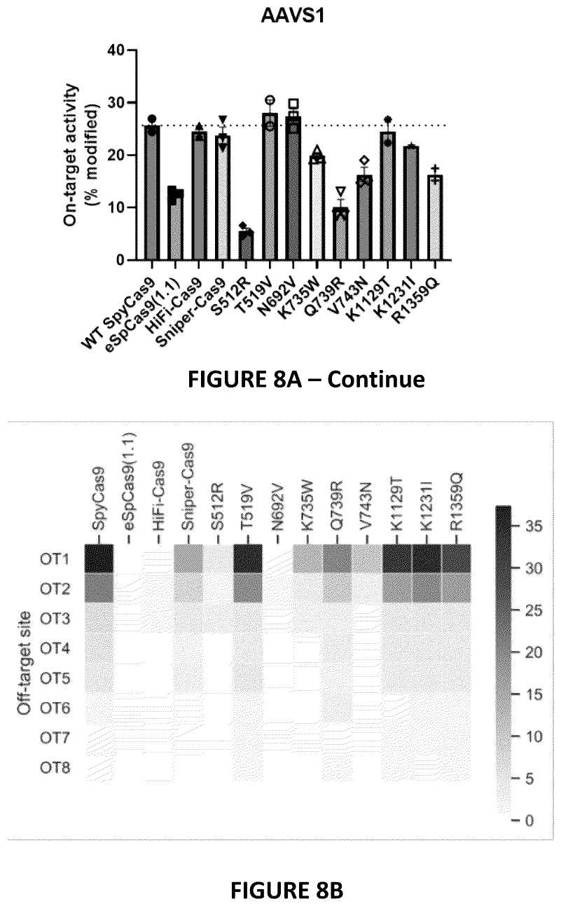

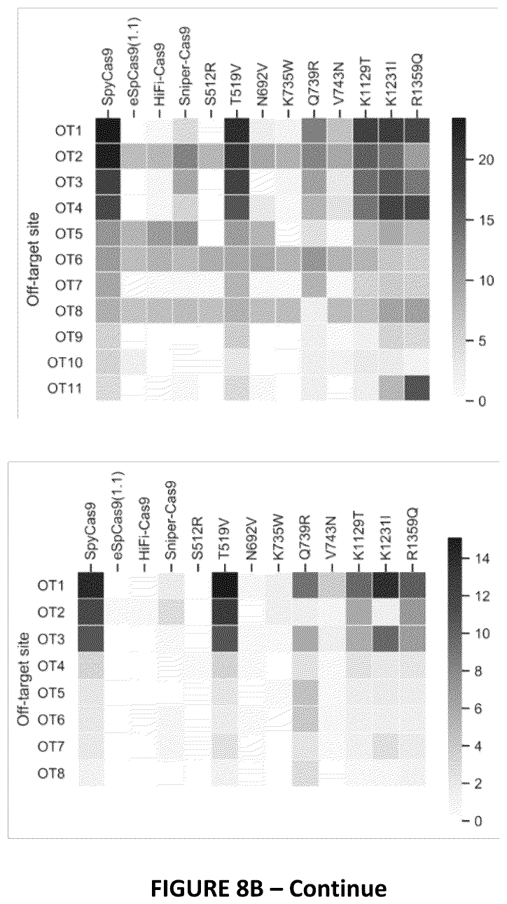

The results show that one of the variants wherein Asparagine at position 692 was substituted with Valine (N692V; Variant #2, FIG. 8) presented HF properties as it had on-target activity similar to wildtype enzyme (i.e., intact activity) while simultaneously demonstrated extremely low off-target editing (e.g., increased specificity).

The in vitro data presented demonstrate and support the feasibility of the herein disclosed in silico directed evolution method for generation of improved macromolecules.

### Example 5

### NMA as the Basis for the ComPE Platform

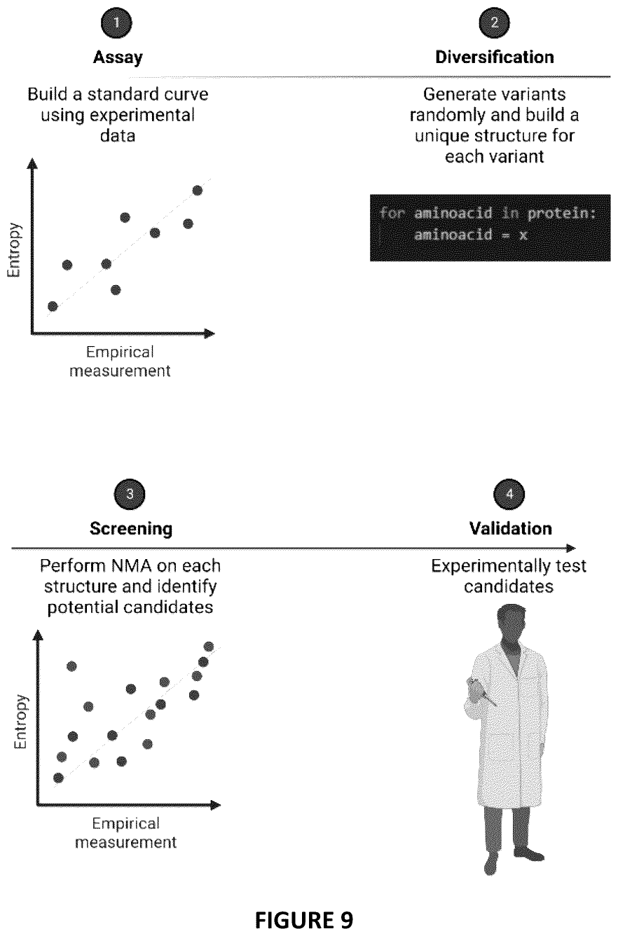

NMA is a dynamic approach to study the function of proteins. The inventors have previously reported the use of NMA to link between genotype and phenotype in context of disease-causing mutations. In addition, the inventors recently reported the use of NMA to assess the specificity profile of SpyCas9 in the presence of mismatches and demonstrated results highly correlative with experimentally data. Inspired by the ability of NMA to simulate the protein function, the inventors used NMA to build ComPE, an in-silico method combining principles of directed evolution and deep mutational scanning (FIG. 9). The first step of the ComPE method, like wet-lab methods, is the assay establishment. While experimental methods relay on survival or reporter-based assays, the inventors used available data of engineered variants to learn the patterns of NMA in relation to experimental empirical data. The second step dictates the nature of diversification: fully or partially random, single, or multiple substitutions, avoidance of active site mutagenesis, substitution to all or limited amino acids and more mutagenesis parameters. This step starts from altering the primary structure of the protein and ends with modeling the allosteric changes in the quaternary structure. Each variant results in a unique structure which will be used by NMA. Next, on the third step, each structure is used as an input for the NMA to assess the entropic profile of the variant and compare it with the data from the first step.

### Example 6

The Assay—Entropy of HF Variants Correlate with the Activity and Specificity

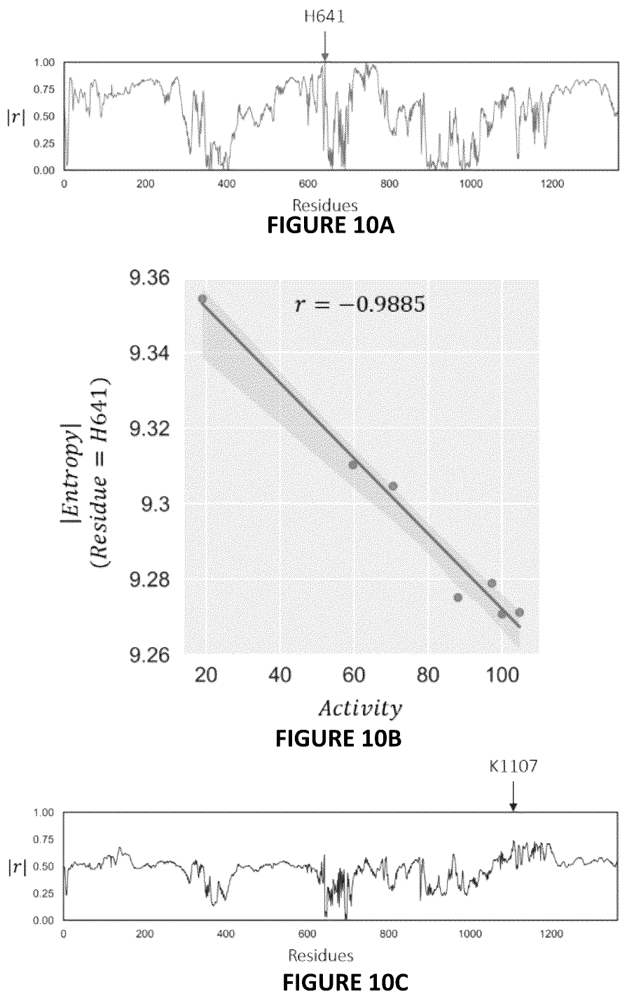

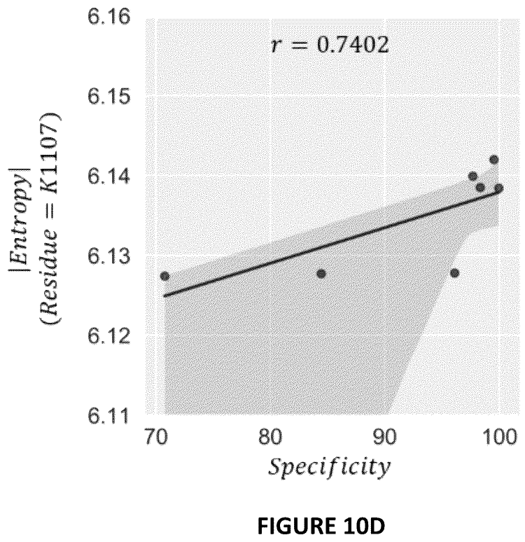

The association between the structure of a protein and its function is the basis for the current ability to extrapolate the properties of the evolving protein. It is commonly accepted to classify the function of Cas9 into on-target activity and specificity. While both parameters are derived from the protein's activity level, there is a clear negative correlation between the two. For example, HF variants with extremely high specificity (i.e., evoCas9), suffer from a severe reduction in on-target activity levels. Since most Cas9 engineered HF variants exhibit impaired on-target activity, the inventors aimed to discover novel variants with improved specificity and intact on-target activity. NMA was utilized to calculate the entropy of SpyCas9 and six HF variants (eSpCas9(1.1), SpCas9-HF1, HiFi-Cas9, HypaCas9, evoCas9 and Sniper-Cas9). The entropy values were compared with experimentally measured values of activity and specificity, as previously reported by Schmid-Burgk et al., (2020). The higher the correlation between the entropy and the empirical data, the accuracy of subsequent predictions is improved. Therefore, the inventors sought to assess the entropy of which residue yields the best correlation. Once a structure was generated for each HF variant (based on the structure of WT SpyCas9, PDB:5F9R), the inventors examined the entropy of which residue returns the highest correlation coefficient (r) for all seven Cas9 enzymes when compared with either the activity or specificity scores (FIG. 10). The selected residue for entropy calculation is not implied to be a candidate for mutagenesis, but rather a reliable indicator of the entropy-function correlation. The inventors found that H641 and K1107 best correlated with the activity (r=−0.9885, FIGS. 10A-10B) and specificity (r=0.7402, FIGS. 10C-10D), respectively. These residues were used for further analyses in the current study. The contrary direction of the activity and specificity correlations represents the tradeoff between the two.

### Example 7

### Diversification and Screening—In-Silico Deep Mutational Scanning and Selection of HF Cas9 Candidates

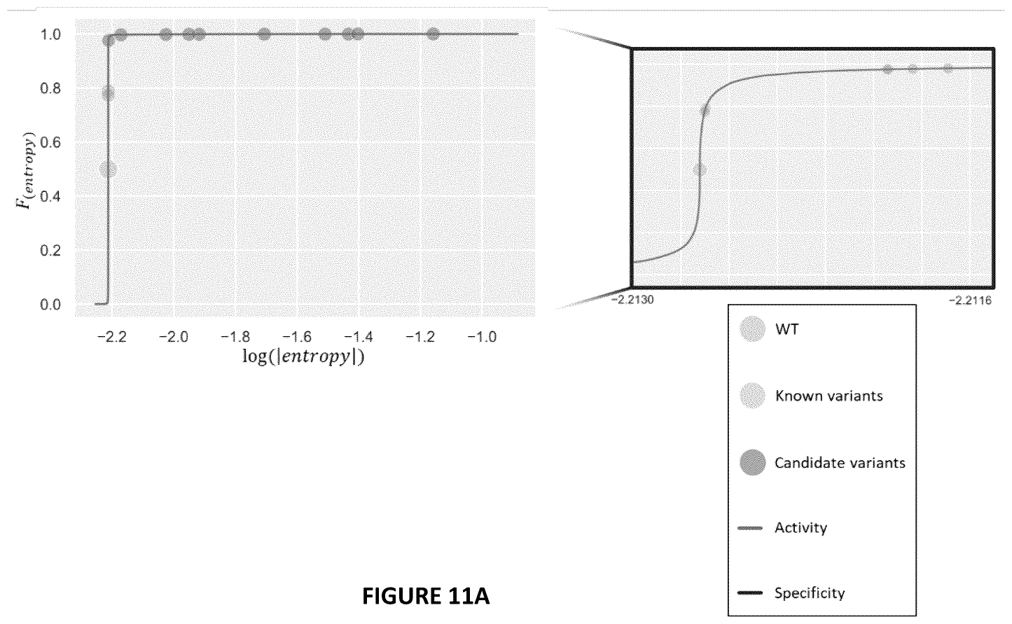

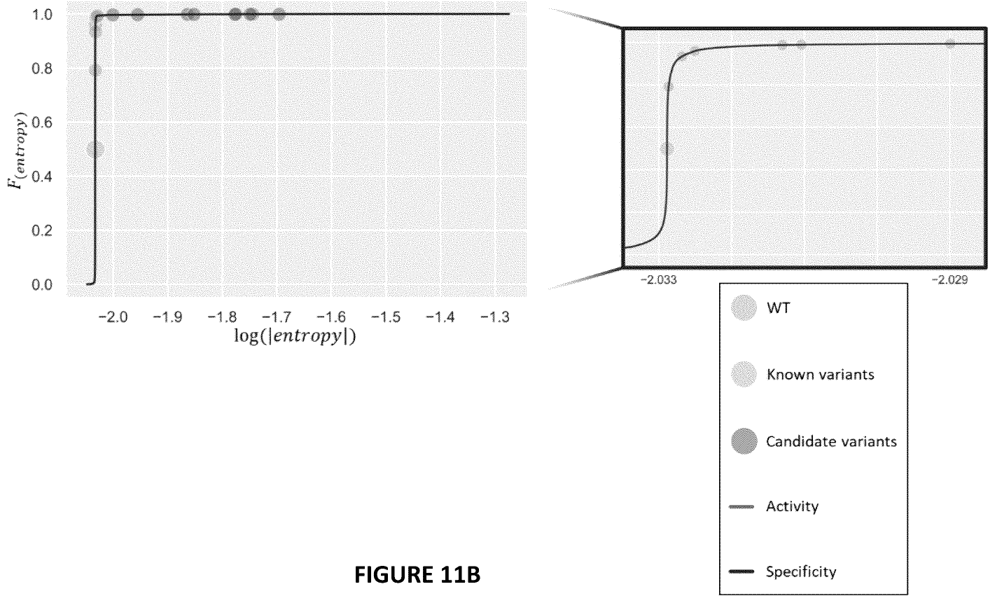

To discover novel single-mutation SpyCas9 with improved specificity, the inventors initiated the diversification process by simply substituting each residue of the protein (1,362 residues) with every other amino acid (19 alternatives). In total, 25,878 new structures were generated and analyzed by NMA to calculate their entropy. For all the variants, the entropy values were plotted on an empirical cumulative distribution function (ECDF) graph. In agreement with the consensus, the inventors observed that most single-mutation substitutions merely altered the entropy of the protein, having most of the variants concentrated around Med_{(Activity)}=−2.213 (FIG. 11A) and Med_{(Activity)}=−2.033 (FIG. 11B). Interestingly, the known HF variants were all positioned above the WT Cas9 for both the activity and specificity, excluding Sniper-Cas9 with lower entropy on the specificity plot. Taken together with the previous results, these data strengthen our hypothesis that moderate destabilization of Cas9, depicted with mild increase of entropy, would lead to improved specificity of the protein. The inventors picked nine candidates, all from the top 0.25% (F_{(entropy)}≥0.9975), similar but with higher entropy than the compared known HF variants, from different regions and domains of the protein. None of the selected mutations are part of any of the existing HF variants. The selected candidates are: S512R, T519V, N692V, K735W, Q739R, V743N, K1129T, K1231I and R1359Q (in a sequence-chronological order). Interestingly, the entropy of WT Cas9 was found to have F_{(entropy)} of ˜0.5 for both activity and specificity. The nature of the WT enzyme is to be stable and specific on one hand, but also adjustable to small changes on the other hand. This observation may imply an evolutionary balance, leading to stability suitable for the function of the enzyme, favorable over other small changes in the sequence of the protein.

### Example 8

HF Cas9 Candidates have Reduced Genome-Wide Off-Targets

Plasmids carrying the nine candidates were constructed, as well as WT SpyCas9, eSpCas9(1.1), HiFi-Cas9 and Sniper-Cas9. All variants were synthesized and cloned into the same backbone, to eliminate differences derived from different plasmid elements (i.e., regulatory elements and codon usage). To characterize the genome-wide off-targeting activity of the current candidates compared to WT SpyCas9 and the known HF variants, the inventors used three sgRNAs targeting different loci, EMX1, VEGFA and AAVS1. Each sgRNA plasmid was co-transfected into HEK293 ft cells with any of the Cas plasmids and genomic DNA was extracted 96 hours post-transfection. The inventors evaluated the on-target and off-targets fractions by analyzing next generation sequencing (NGS) data of the rhAmpSeq assay, a method for on and off-targets evaluation by multiplex PCR and NGS. Primers design for multiplex PCR was based on previously reported off-targets data for these particular gRNA sequences in the same cell line. Off-target sites for EMX1 and VEGFA site 3 were identified using GUIDE-Seq and TTISS by Schmid-Burgk et al., and off-targets for the AAVS1 gRNA were identified using GUIDE-Seq by Integrated DNA Technologies (IDT). The analysis of the raw NGS data was performed using CRISPResso2. First, the inventors sought to assess whether the tested variants are characterized by poor on-target activity compared to WT SpyCas9 (FIG. 8A), as many of the engineered HF variants are known to have reduced levels of activity. Indeed, the inventors have observed decreased levels of activity in eSpCas9(1.1) and some of the current candidates. Yet, N692V was found to have intact levels of on-target activity. The inventors further analyzed the off-target activity of the variants and identified four predicted variants (S512R, N692V, K735W and V743N) with compelling reduced off-target activity. Out of the novel HF variants, N692V is the only one to demonstrate both intact on-target activity with reduced off-targeting, pointing it as a prominent HF Cas variant.

### Example 9

### Single-Base Resolution—Specificity Profile of the HF Cas9 Candidates

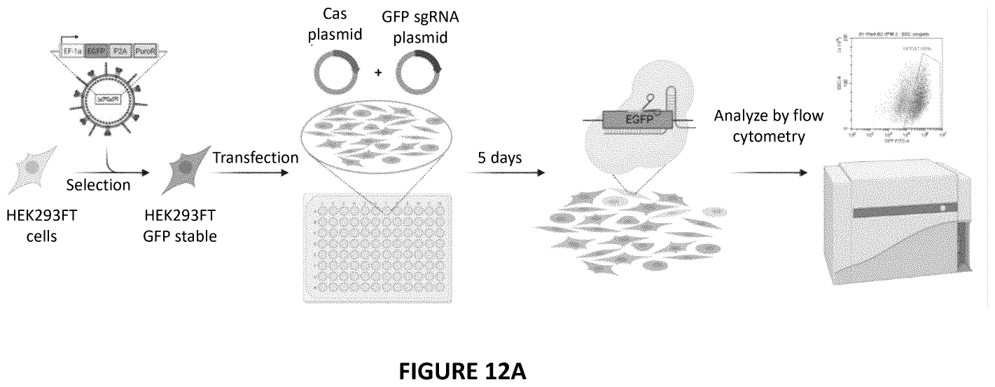

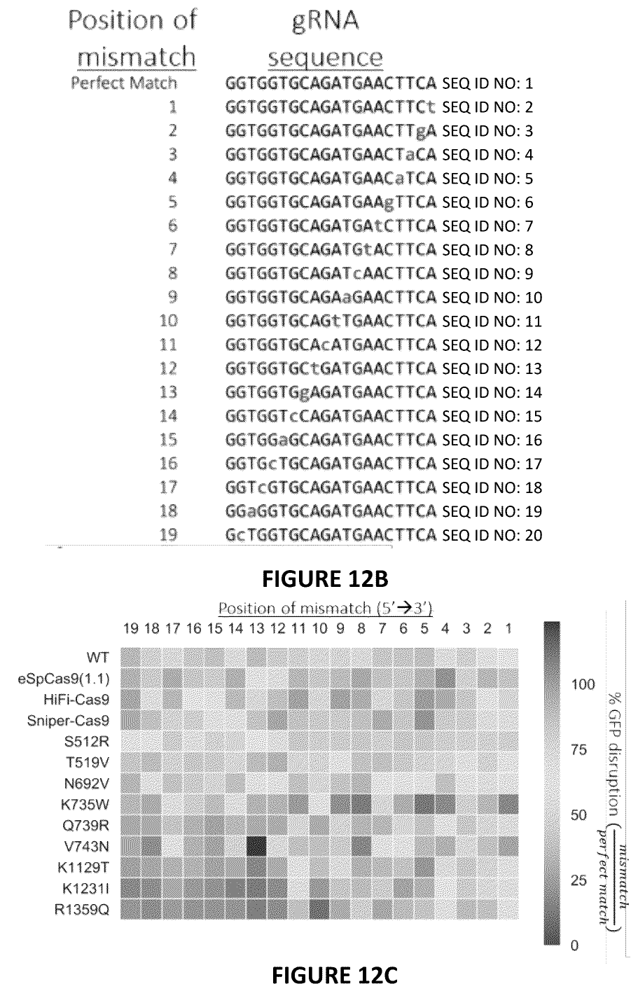

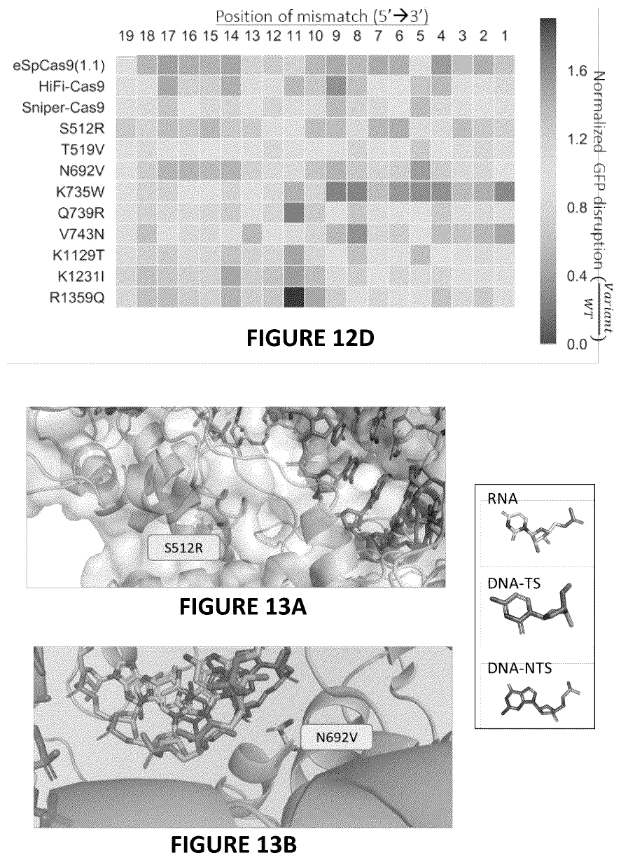

Previous studies have demonstrated the specificity profile of Cas enzymes by different methods. High-throughput methods are generally based on either flow cytometry or deep sequencing. In contrast to genome-wide off-targets analysis, the specificity profile describes the single base resolution of Cas enzymes. It offers a deeper understanding of the mutational effects on the engineered variants, and it may explain the off-targeting patterns observed in unbiased methods such as rhAmpSeq, GUIDE-seq and others. Motivated by the rhAmpSeq off-targets analysis, the inventors employed the GFP disruption assay, using sgRNAs targeting the GFP gene with mismatches in each of the positions within the gRNA. By transfecting plasmids carrying a Cas enzyme and an sgRNA into EGFP-stable HEK293 ft cells, the activity of Cas9 can be measured as the decrease of GFP fluorescence intensity using flow cytometry (FIG. 12A). Each variant was co-transfected with 20 different sgRNAs. One sgRNA plasmid has the perfect match sequence to assess the maximal GFP reduction, while the other 19 plasmids carry an sgRNA with a transversion mismatch (A↔T and G↔C) in positions 1-19. Since the hU6 promoter which drives the expression of the sgRNA, requires a 5′G to initiate transcription, the 20^{th} position remained unaltered (FIG. 12B).

In consistency with the rhAmpSeq results (FIG. 8), the four variants S512R, N692V, K735W and V743N, were once again identified as HF variants with improved sensitivity to mismatches (FIG. 12C). Furthermore, the inventors found different patterns of specificity between two groups of variants: S512R and N692V were improved all along the gRNA, while K735W and V743N showed HF properties along the seed region of the gRNA (positions one through eleven). It is noteworthy that residues 512 and 692 are part of the REC III domain (REC lobe), while residues 735 and 743 lay within the RuvC II domain (NUC lobe) of Cas9. Mutations of residues at both regions are commonly abundant in the variety of engineered Cas9 variants. Furthermore, the inventors compared each variant with WT Cas9 to assess its improvement (FIG. 12D). The improvement of S512R and N692V was comparable with the other known HF variants the inventors tested. Surprisingly, although being partially HF, K735W demonstrated increased specificity compared to all other tested variants within the positions of the seed sequence. Taken together, the results presented herein demonstrate the improvement of four new computationally predicted variants, S512R, N692V, K735W and V743N, compared to WT Cas9 and three known HF Cas9 variants.

### Discussion

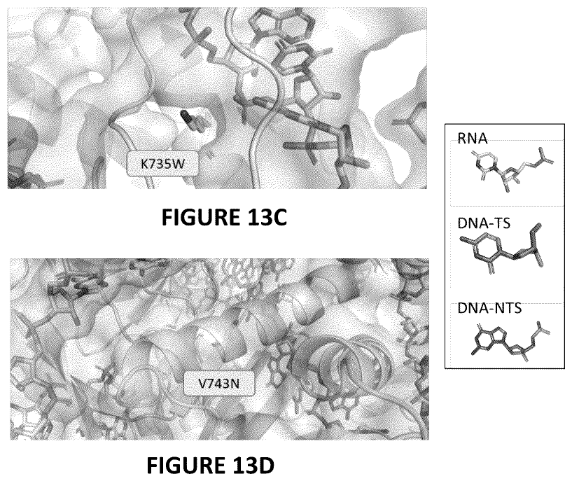

Presented herein is ComPE, a novel approach for computational entropy-based deep mutational scanning. The inventors employed ComPE to engineer SpyCas9 variants with improved specificity. While previously described Cas9 HF variants were engineered either by rational design or experimental directed evolution, here the inventors report for the first time the use of an unbiased in-silico assay to predict and generate engineered variants with improved specificity. Four (out of nine) of the currently predicted candidates were found to have improved specificity by experimental assays, pointing them as successful HF variants. Notably, N692V also demonstrated intact on-target activity levels. As CRISPR-based therapeutics show promising results in clinical trials and move forward toward applicable solutions to treat genetic conditions, engineered Cas enzymes have a great potential in increasing the safety of such treatments by reducing unintended off-target activity. Previous studies demonstrated that NMA could be utilized to analyze the effect of point mutations on proteins in terms of stability and flexibility. However, here, we report for the first time, the use of NMA to perform deep mutational scanning, based on reference experimental data, to improve a protein's function in terms of binding specificity to binding counterparts (i.e., DNA, RNA, or other proteins). As binding properties can be described using entropy, the inventors believe that the current approach can be helpful for engineering different biological macromolecules, and the interactions between them. It is noteworthy that the mutations the inventors report, as predicted by the current method, were non-trivial substitutions between different groups of amino acids with distinct properties (S512R, N692V, K735W and V743N). Moreover, out of the four identified point mutations, two were found to be in close proximity to the gRNA or DNA (N692 and K735), while the other two (S512 and V743) are not in close contact with nucleic acid(s) in the structure (FIG. 13). It is likely that allosteric effects account for the modified function of the protein. This observation emphasizes the impact of the current method, similar to random directed evolution, but different from rational design, the inventors were able to identify beneficial mutations remote from the active site or ligand-binding residues. Another advantage of the current computational mutagenesis approach is that the mutations are installed in the protein level (i.e., mutagenesis of amino acids in the structure), where in experimental directed evolution, the mutagenesis takes place in the DNA level. Thus, mutations such as those the inventors describe here (N↔V, T→V and K→W), which require the alteration of two nucleotides in the codon, are less likely to arise in an experimental directed evolution campaign. Currently, this method is limited by computational resources and inefficient processes. It can be assumed that in the future, advanced capabilities will enable multiple iterations of mutagenesis, enabling computational directed evolution and prediction of more complex combinatorial mutagenesis. Furthermore, another limitation is the requirement of a protein structure. This limitation could have been potentially solved by recent advances in protein structure prediction. However, these and other tools are yet to be able to predict the structure of a complex with multiple counterparts (e.g., SpyCas9 bound to its sgRNA and target DNA), and their interaction. Such capabilities, combined with entropy-based methods, would greatly advance the field of computational protein engineering and drug discovery.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for identifying a reference macromolecule for which a variant having improved function can be engineered, the method comprising:
a. receiving a dataset comprising data on relative functionality of:
i. variants of said reference macromolecule comprising an altered residue or moiety;
ii. said reference macromolecule in complex with different binding counterparts; or
iii. both;
b. in silico calculating a value of entropy for said variants of said reference macromolecule and/or said reference macromolecule in complex with different binding counterpart of said received dataset;
c. determining a correlation value between said calculated values of entropy and said received relative functionality data; wherein a correlation value above a predetermined threshold indicates a true correlation between entropy and function; and
d. identifying a reference macromolecule with a true correlation between entropy and function as a reference macromolecule for which a variant having improved function can be engineered.

2. The method of claim 1, wherein said dataset of step (a) comprises data on relative functionality of said reference macromolecule in complex with different binding counterparts and said method further comprises as part of step (b) in silico calculating a value of entropy for said different binding counterparts in complex with said reference macromolecule and as part of step (c) determining a correlation value between said calculated value of entropy of said different binding counterparts and said received functionality data, wherein a correlation value of the macromolecule entropy above a predetermined threshold and a correlation value of the binding counterpart entropy above a predetermined thresholds indicates a true correlation.

3. The method of claim 1, wherein said dataset of step (a) comprises data on relative functionality of said reference macromolecule in complex with different binding counterparts and said method further comprises:
receiving a second dataset comprising data on relative functionality of variants of said reference macromolecule comprising an altered residue or moiety,
in silico calculating a value of entropy for said variants of said reference macromolecule,
determining a correlation value between said calculated values of entropy of said altered macromolecules and said relative functionality data of said second dataset;
and wherein step (d) comprises selecting a reference macromolecule with true correlation based on said dataset of step (a) and said second dataset.

4. The method of claim 1, wherein said correlation is a Pearson correlation coefficient (R), and optionally wherein said above a predetermined threshold is above an R of 0.55.

5. (canceled)

6. The method of claim 1, wherein; (i) said calculating a value of entropy comprises normal mode analysis (NMA); (ii) said correlation is a positive correlation or a negative correlation and said correlation value is an absolute value of said correlation; or both (i) and (ii).

7. (canceled)

8. The method of claim 1, wherein said value of entropy is an absolute value of entropy.

9. The method of claim 1, wherein said macromolecule is a protein, a polynucleotide, or a complex comprising both.

10. The method of claim 1, wherein said macromolecule is a protein, and wherein said functionality is selected from the group consisting of: thermostability, conformational transition, binding to a substrate, enzymatic activity, and signaling, and optionally wherein said protein is an enzyme, said functionality in enzymatic activity or binding and said substrate is a target of said enzymatic activity.

11. (canceled)

12. The method of claim 1, wherein said binding counterpart is a protein or nucleic acid molecule that forms a complex with said reference macromolecule by direct binding or binding via an intermediate molecule, and optionally wherein: (i) said intermediate molecule is a nucleic acid molecule that binds a binding counterpart that is a nucleic acid molecule; (ii) said binding counterpart, said intermediate molecule or both is a nucleic acid molecule selected from DNA and RNA; or (iii) both (i) and (ii).

13.-14. (canceled)

15. The method of claim 9, wherein said protein is a genome-editing protein, optionally wherein said genome-editing protein is a CRISPR associated (Cas) protein, and optionally wherein said binding counterpart is a target genomic locus, said intermediate molecule is a guide RNA (gRNA) and said value of entropy is calculate for any one of: said Cas protein alone, said Cas protein complexed with a gRNA and said Cas protein complexed with a gRNA and said target genomic locus.

16. (canceled)

17. A method for engineering a variant of a macromolecule having improved function as compared to a reference macromolecule, the method comprising:
a. identifying a reference macromolecule suitable for engineering by according to the method of claim 1;
b. generating a standard curve of entropy value to function based on said calculated entropy values and said received relative functionality data;
c. in silico calculating a value of entropy for at least one new variant of said reference macromolecule;
d. based on said generated standard curve and said calculated value of entropy predicting relative function of said at least one new variant; and
e. selecting a new variant with predicted improved relative function as compared to said reference macromolecule;
thereby engineering a variant of a macromolecule having improved function as compared to a reference macromolecule, and optionally wherein: (i) said method further comprises synthesizing said selected macromolecule variant engineered to have improved function as compared to said reference macromolecule; (ii) said method further comprises determining said synthesized macromolecule variant has improved function as compared to said reference macromolecule, wherein said determining is performed in vitro, in vivo, ex vivo, or any combination thereof: or (iii) both (i) and (ii).

18.-19. (canceled)

20. A macromolecule variant engineered or synthesized according to the method of claim 17, optionally wherein said macromolecule being a protein, a polynucleotide, or a complex comprising at least one protein and at least one polynucleotide, and optionally wherein said polynucleotide comprises DNA, RNA, or a hybrid thereof.

21.-22. (canceled)

23. A Cas variant protein of a reference Cas protein wherein said reference Cas protein comprises an amino acid sequence as set forth in (SEQ ID NO: 1), wherein said variant protein comprises at least one amino acid substitution in a position selected from the group consisting of: 692, 1129, 1231, 9, 519, 177, 381, 395, 414, 512, 538, 539, 735, 739, 743, 758, 871, 1256, 1283 1359, and any combination thereof, in said reference Cas protein, optionally wherein said reference Cas protein is a Streptococcus pyogenes Cas wildtype (SpyCas) protein, and optionally wherein said SpyCas is SpyCas9.

24.-25. (canceled)

26. The Cas variant protein of claim 23, characterized by having improved function compared to said Cas reference protein, and optionally wherein said improved function is selected from improved substrate specificity and improved nuclease activity.

27. (canceled)

28. The Cas variant protein of claim 23, wherein said at least one amino acid substitution is selected from the group consisting of: N692V, K1129T, K1231I, L9R, T519V, D177A, E381N, R395A, I414A, S512R, A538I, F539V, K735W, Q739R, V743N, N758V, P871I, Q1256W, A1283E, R1359Q, and any combination thereof, and optionally wherein said at least one amino acid substitution is selected from the group consisting of: N692V, S512R, K735W, V743N, and any combination thereof.

29. (canceled)

30. A nucleic acid molecule comprising a nucleic acid sequence encoding the Cas variant protein of claim 23.

31. An expression vector comprising the nucleic acid sequence of claim 30.

32. A cell comprising the nucleic acid sequence of claim 30.

33. A composition comprising the cell of claim 32 and an acceptable carrier, and optionally wherein said composition is a pharmaceutical composition.

34. (canceled)

35. A method for modifying at least one target nucleic acid sequence of interest, the method comprising contacting said at least one target nucleic acid sequence of interest with an effective amount of:
a. the Cas variant protein of claim 23; and
b. at least one target recognition element or a nucleic acid sequence encoding thereof,
thereby modifying the at least one target nucleic acid sequence of interest, and optionally wherein said target nucleic acid sequence of interest is in cell of a subject, and said contacting is administering a therapeutically effective to said subject.

36. (canceled)


SEQUENCE LISTING (22 sequences)

SEQ ID NO: 1 - protein, 1368 aa, synthetic construct [sequence omitted]

SEQ ID NO: 2 - protein, 1368 aa, synthetic construct
  VAR_SEQ 9: note = X is any amino acid except Leucine
  VAR_SEQ 177: note = X is any amino acid except Aspartic acid
  VAR_SEQ 381: note = X is any amino acid except Glutamic acid
  VAR_SEQ 395: note = X is any amino acid except Arginine
  VAR_SEQ 414: note = X is any amino acid except Isoleucine
  VAR_SEQ 512: note = X is any amino acid except Serine
  VAR_SEQ 519: note = X is any amino acid except Threonine
  VAR_SEQ 538: note = X is any amino acid except Alanine
  VAR_SEQ 539: note = X is any amino acid except Phenylalanine
  VAR_SEQ 692: note = X is any amino acid except Asparagine
  VAR_SEQ 735: note = X is any amino acid except Lysine
  VAR_SEQ 739: note = X is any amino acid except Glutamine
  VAR_SEQ 743: note = X is any amino acid except Valine
  VAR_SEQ 871: note = X is any amino acid except Proline
  VAR_SEQ 758: note = X is any amino acid except Asparagine
  VAR_SEQ 1129: note = X is any amino acid except Lysine
  VAR_SEQ 1231: note = X is any amino acid except Lysine
  VAR_SEQ 1256: note = X is any amino acid except Glutamine
  VAR_SEQ 1283: note = X is any amino acid except Alanine
  VAR_SEQ 1359: note = X is any amino acid except Arginine
MDKKYSIGXDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPXNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILXKMDGTEELLVKLNXEDLLRKQRTFDNGSIPHQXHLGELHAILRRQEDFYPFLKDNREKIEKILTFRIPYYVGPLARGNSRFAWMTRKSEETITPWNFEEVVDKGASAQSFIERMTNFDKNLPNEKVLPKHXLLYEYFXVYNELTKVKYVTEGMRKPXXLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRXFMQLIHDDSLTFKEDIQKAQVSGQGDSLHEHIANLAGSPAIKXGILXTVKXVDELVKVMGRHKPEXIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVXSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPXKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSXYVNFLYLASHYEKLKGSPEDNEQKXLFVEQHKHYLDEIIEQISEFSKRVILXDANLDKVLSAYNKHRDKPIREQAENIIHLFTLTNLGAPAAFKYFDTTIDRKRYTSTKEVLDATLIHQSITGLYETXIDLSQLGGD

SEQ ID NO: 3 - protein, 1368 aa, synthetic construct
  VAR_SEQ 9: note = X is any amino acid except Leucine
MDKKYSIGXDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, synthetic construct
  VAR_SEQ 177: note = X is any amino acid except Aspartic acid
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPXNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, synthetic construct
  VAR_SEQ 381: note = X is any amino acid except Glutamic acid
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILXKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1368 aa, synthetic construct
  VAR_SEQ 395: note = X is any amino acid except Arginine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNXEDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1368 aa, synthetic construct
  VAR_SEQ 414: note = X is any amino acid except Isoleucine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQXHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1368 aa, synthetic construct
  VAR_SEQ 512: note = X is any amino acid except Serine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHXLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1368 aa, synthetic construct
  VAR_SEQ 519: note = X is any amino acid except Threonine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFXVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - protein, 1368 aa, synthetic construct
  VAR_SEQ 538: note = X is any amino acid except Alanine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPXFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 1368 aa, synthetic construct
  VAR_SEQ 539: note = X is any amino acid except Phenylalanine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAXLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1368 aa, synthetic construct
  VAR_SEQ 692: note = X is any amino acid except Asparagine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRXFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 13 - protein, 1368 aa, synthetic construct
  VAR_SEQ 735: note = X is any amino acid except Lysine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKXGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1368 aa, synthetic construct
  VAR_SEQ 739: note = X is any amino acid except Glutamine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILXTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - protein, 1368 aa, synthetic construct
  VAR_SEQ 743: note = X is any amino acid except Valine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKXVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 16 - protein, 1368 aa, synthetic construct
  VAR_SEQ 758: note = X is any amino acid except Asparagine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPEXIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 1368 aa, synthetic construct
  VAR_SEQ 871: note = X is any amino acid except Proline
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVXSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 1368 aa, synthetic construct
  VAR_SEQ 1129: note = X is any amino acid except Lysine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPXKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - protein, 1368 aa, synthetic construct
  VAR_SEQ 1231: note = X is any amino acid except Lysine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSXYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - protein, 1368 aa, synthetic construct
  VAR_SEQ 1256: note = X is any amino acid except Glutamine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKXLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1368 aa, synthetic construct
  VAR_SEQ 1283: note = X is any amino acid except Alanine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILXDANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 1368 aa, synthetic construct
  VAR_SEQ 1359: note = X is any amino acid except Arginine
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETXIDLSQLGGD